(19) United States
(12) Patent Application Publication
(10) Pub. No.: US 2004/0209953 A1
(43) Pub. Date: Oct. 21, 2004

(54) Glyceride compositions and methods of making and using same

(76) Inventor: Theresa Siu-Ling Wai Lee, Upper Arlington, OH (US)

Correspondence Address: Ross Products Division, Abbott Laboratories, 625 Cleveland Avenue, Columbus, OH, 43221, US

(21) Appl. No.: 10/705,080

(22) Filed: Nov. 10, 2003

Related U.S. Application Data
(60) Provisional application No. 60/431,338, filed on Dec. 6, 2002.

(51) Int. Cl. A61K 31/225

(52) U.S. Cl. 514/547

(57) ABSTRACT

Disclosed are glyceride compositions, methods of making the glyceride compositions, and nutritional formulations containing the glyceride compositions. The glyceride compositions contain predominantly monoglycerides and diglycerides carrying one or more long chain polyunsaturated fatty acids. Also disclosed are methods of using the glyceride compositions and nutritional formulations.

### CROSS REFERENCE

This application is related to the Glyceride Compositon and Methods of Making and Using Same which was filed as Ser. No. 60/431,338 on Dec. 6, 2002.

### TECHNICAL FIELD

The present invention generally relates to glyceride compositions containing monoglycerides and/or diglycerides of long chain polyunsaturated fatty acids, nutritional formulations containing the monoglycerides and/or diglycerides of long chain polyunsaturated fatty acids, and various methods associated with making and using the glyceride compositions and nutritional formulations.

### BACKGROUND ART

Long chain polyunsaturated fatty acids (LCPUFAs) are essential to the human diet. Two of the families of LCPUFAs include the omega 3 fatty acids, exemplified by docosahexaenoic acid (DHA), and the omega 6 fatty acids, exemplified by arachidonic acid (AA). LCPUFAs are important components of the plasma membrane of the cell, where they may be found in such forms as phospholipids and triglycerides. LCPUFAs are necessary for proper development, particularly in the developing infant brain, in treatment of heart disease, and for tissue formation and repair. LCPUFAs also serve as precursors to other molecules of importance in human beings and animals, including the prostacyclins, eicosanoids, leukotrienes and prostaglandins. Six major long chain LCPUFAs of importance include DHA and eicosapentaenoic acid (EPA), gamma-linolenic acid (GLA), arachidonic acid (AA), linoleic (LA) and alpha-linolenic (ALA).

Several disorders respond to treatment with LCPUFAs. For example, supplementation with LCPUFAs has been shown to reduce the rate of restenosis after angioplasty. Fish oil supplements have been shown to improve symptoms of inflammation and rheumatoid arthritis, and LCPUFAs have been suggested as treatments for asthma and psoriasis. Some evidence indicates that LCPUFAs may be involved in calcium metabolism, suggesting that LCPUFAs may be useful in the treatment or prevention of osteoporosis and of kidney or urinary tract stones.

LCPUFAs can be used in the treatment of cancer. Malignant cells have been shown to have altered fatty acid compositions; addition of fatty acids has been shown to slow their growth and cause cell death, and to increase their susceptibility to chemotherapeutic agents. GLA has been shown to cause reexpression in cancer cells of the E-cadherin cellular adhesion molecules, loss of which is associated with aggressive metastasis. Clinical testing of intravenous administration of the water soluble lithium salt of GLA to pancreatic cancer patients produced statistically significant increases in their survival. LCPUFA supplementation may also be useful for treating cachexia associated with cancer.

LCPUFAs also can be used to treat diabetes. Altered fatty acid metabolism and composition has been demonstrated in diabetic animals. These alterations have been suggested to be involved in some of the long-term complications resulting from diabetes, including retinopathy, neuropathy, nephropathy and reproductive system damage. Primrose oil, which contains GLA, has been shown to prevent and reverse diabetic nerve damage.

Essential fatty acid deficiency has been suggested as being involved in eczema, and studies have shown beneficial effects on eczema from treatment with GLA. GLA has also been shown to reduce increases in blood pressure associated with stress, and to improve performance on arithmetic tests. GLA has been shown to inhibit platelet aggregation, cause vasodilation, lower cholesterol levels and inhibit proliferation of vessel wall smooth muscle and fibrous tissue. Administration of GLA, alone or in combination with EPA, has been shown to reduce or prevent gastrointestinal bleeding and other side effects caused by non-steroidal anti-inflammatory drugs. GLA has also been shown to prevent or treat endometriosis and premenstrual syndrome and to treat myalgic encephalomyelitis and chronic fatigue after viral infections.

AA and DHA are important elements of muscle, organ, blood, and vascular tissues. AA serves a major role as a structural lipid associated predominantly with phospholipids in blood, liver, muscle and other major organ systems. In addition to its primary role as a structural lipid, AA also is the direct precursor for a number of circulating eicosenoids such as prostaglandin E₂ (PGE₂), prostacyclin I₂ (PGI₂), thromboxane A₂ (TₓA₂), and leukotirenes B₄ (LTB₄) and C₄ (LTC₄). These eicosenoids exhibit regulatory effects on lipoprotein metabolism, blood rheology, vascular tone, leucocyte function and platelet activation. The prostaglandins are known to influence conditions such as blood clotting, inflammatory and anti-inflammatory response, cholesterol absorption, bronchial function, hypertension, visual acuity and brain development in infants, and gastric secretions, among other effects.

The composition of human milk serves as a valuable reference for improving infant formula. Much effort is directed at producing a milk based infant formula which is similar to human milk. Human milk fat contains LCPUFAs, which play a role in infant development. In human milk, about 98% of the fatty acids are in triglycerides. However, infant formulas may not contain lipids having LCPUFAs such as AA, EPA, and DHA.

Polyunsaturated acids, in particular the longer chain acids such as AA, DHA and EPA, are natural constituents of many foodstuffs. These LCPUFAs are either intimately combined with less desirable components such as cholesterol, phosphorus compounds, or are unsuitable for food applications in their functional form. Acceptable ingredient sources for these LCPUFAs are limited, thus the absence of such components in infant formula and adult nutritionals.

Some LCPUFAs, such as AA, cannot be synthesized de novo in humans. AA is synthesized by the enzymatic elongation and desaturation of linoleic acid (LA), which is related to GLA. LA, in turn, is an essential acid which can only be obtained from dietary sources. To complicate matters, the presence of EPA in the diet inhibits the metabolic conversion of LA to AA.

During the first year of its life, an infant can double or triple its birth weight. This rapid tissue growth phase may be enhanced by elevated levels of dietary AA. In addition, human breast milk contains relatively high levels of AA. Some mothers who breast-feed their infants may benefit from additional dietary AA, particularly vegetarians. However, many mothers do not breast feed their infants, or do not breast feed for the entire period of rapid infant growth, choosing instead to utilize an infant formula. There is a need to improve the availability of LCPUFAs in nutritional formulations including infant formulae.

One difficulty of including LCPUFA in nutritional formulations is the high susceptibility of many LCPUFAs to oxidation, particularly ALA, GLA, AA, EPA, and DHA. Oxidation of LCPUFAs causes rancidity and deterioration of the nutritional and organoleptic properties of the nutritional products. Such oxidation occurs at elevated temperatures and/or in packaging when exposed to air and oxygen environment. Control methods against lipid oxidation include the use of metal inactivators, minimizing the exposure to air and light during processing, preventing the loss of natural antioxidants, addition of natural and synthetic antioxidants, and storage at refrigerated and frozen temperatures. The effectiveness of these methods is quite limited and is financially costly. Thus, there remains a need to significantly improve the oxidation stability of food and nutritional products containing LCPUFA.

### SUMMARY OF THE INVENTION

The following is a summary of the invention in order to provide a basic understanding of some aspects of the invention. This summary is not intended to identify key/critical elements of the invention or to delineate the scope of the invention. Its sole purpose is to present some concepts of the invention in a simplified form as a prelude to the more detailed description that is presented later.

One aspect of the present invention relates to glyceride compositions and methods of preparing a chemically synthesized mixture of monoglycerides and diglycerides (MGDG) of LCPUFAs in which the concentrations and profiles of the fatty acids are similar to the starting materials (triglycerides and phospholipids), while the oxidative stability of the LCPUFA in the monoglycerides and diglycerides mixtures are greatly improved compared to the LCPUFA in the triglycerides and phospholipids of the starting materials. The glyceride compositions may be used in nutritional products. Advantages include those in which one or more of: the nutritional value of the LCPUFA is better preserved, the harmful health effects of oxidized fatty acids and other oxidation byproducts is reduced, the deterioration of the organoleptic properties is reduced and/or eliminated, and the shelf-life of the nutritional products is prolonged. In addition, since LCPUFAs are presented as monoglycerides and diglycerides, the first step of digestion and absorption of a typical dietary fat (hydrolysis by pancreatic lipase) is circumvented. The MGDG-LCPUFA preparation of this invention provides an improved dietary source beneficial in facilitating faster absorption and may be beneficial to people with compromised lipase activity.

To the accomplishment of the foregoing and related ends, the invention comprises the features hereinafter fully described and particularly pointed out in the claims. The following description and the annexed drawings set forth in detail certain illustrative aspects and implementations of the invention. These are indicative, however, of but a few of the various ways in which the principles of the invention may be employed. Other objects, advantages and novel features of the invention will become apparent from the following detailed description of the invention when considered in conjunction with the drawings.

### BRIEF SUMMARY OF THE DRAWINGS

FIG. 1 shows the separation of Monoglyceride (MG), Diglyceride (DG), Triglyceride (TG) and FFA of TG-DHA, FFA-DHA, MGDG-DHA, TG-AA, FFA-AA, MGDG-AA starting oils and preparations by Thin Layer Chromatography. Silica Gel thin layer is used as stationary phase and Chloroform:Acetone:Glacial Acetic Acid (90:10:0.5 by volume) solvent system is used as mobile phase.

FIG. 2 shows a comparison of oxidative stability of TG-DHA and MGDG-DHA (non-RBD).

FIG. 3 shows a comparison of oxidative stability of TG-AA and MGDG-AA (non-RBD).

FIG. 4 shows the separation of MG, DG, TG and FFA of TG-AASOY oil mixture, FFA-AASOY, MGDG-AASOY preparations by Thin Layer Chromatography. Silica gel thin layer is used as stationary phase and Chloroform:Acetone:Glacial Acetic Acid (90:10:0.5 by volume) solvent system is used as mobile phase.

FIG. 5 shows the analysis of FFA-Preparations of Salmon, Cod-Liver, and Borage Oils by Thin Layer Chromatography. Silica gel thin layer is used as stationary phase and Hexane:Ethyl Ether:Glacial Acetic Acid (80:20:1 by volume) solvent system is used as mobile phase.

FIG. 6 shows the analysis of Neutral Lipid Classes of MGDG Preparations of Salmon, Cod-Liver, and Borage Oils by Thin Layer Chromatography. Silica gel thin layer is used as stationary phase and Hexane:Ethyl Ether:Glacial Acetic Acid (80:20:1 by volume) solvent system is used as mobile phase.

### DISCLOSURE OF INVENTION

Terminology

There are numerous parameters and ranges described herein, often with cascading figures. In any parameter or range, the low or high figure may be combined with a low/high figure from another parameter or range. Moreover, each range implicitly includes each integer with the range, which may constitute a high or low figure.

Unless the context indicates otherwise, the following terms shall have the following meaning:

“AA” is arachidonic acid (C20:4n-6); “DHA” is docosahexaenoic acid (C22:6n-3); “EPA” is eicosapentaenoic acid (20:5n-3); “SDA” stearidonic acid (18:4n-3); LA linoleic acid (18:2n-6); “ALA” is alpha linolenic acid (18:3n-3); “GLA” is gamma linolenic acid (18:3n-6);

“LCPUFA” are long chain polyunsaturated fatty acids, fatty acids with about 18 or more carbon acyl chain and about 2 or more double bonds;

“TG Oil” is processed natural ingredients wherein at least about 90% by weight of the fatty acid is present in the oil as a triacylglycerol;

“Egg Yolk Powder” or “EYP” is freeze dried powder of egg yolk where the egg yolks are isolated from the egg whites from whole eggs and then the water content is removed using a lyophilization process;

“FFA” is free fatty acid; “FFA Preparation” contains at least about 90% of free fatty acids that are unesterified; “FAP” is fatty acid profile; “FAME” is fatty acid lower alkyl esters (lower meaning 1 to 5 carbons);

“MGDG preparation” or “MGDG composition” often used interchangeably contains at least about 70% of the fatty acids as monoacylglycerides and diacylglycerides;

the terms “triacylglycerol” or “triglyceride” are used interchangeably;

the terms “diacylglycerol” or “diglyceride” are used interchangeably; and

the terms “monoacylglycerol” or “monoglyceride” are used interchangeably.

Fatty acids are carboxylic acids and are classified based on the length and saturation characteristics of the carbon chain. Short chain fatty acids have 2 to about 6 carbons and are typically saturated. Medium chain fatty acids have from about 6 to about 16 carbons and are also typically saturated. Long chain fatty acids have from about 18 to about 24 or more carbons and may also be saturated or unsaturated. In longer fatty acids there may be one or more points of unsaturation, giving rise to the terms “monounsaturated” and “polyunsaturated”, respectively. For example, LCPUFAs can have about 18 or more carbons, including about 20 or more carbons.

LCPUFAs are categorized according to the number and position of double bonds in the fatty acids according to a nomenclature well understood by the biochemist. There are two series or families of LCPUFAs, depending on the position of the double bond closest to the methyl end of the fatty acid: the n-3 series contains a double bond at the third carbon, while the n-6 series has no double bond until the sixth carbon. For example, AA has a chain length of 20 carbons and 4 double bonds beginning at the sixth carbon. As a result, it is referred to as “20:4 n-6”. Similarly, DHA has a chain length of 22 carbons with 6 double bonds beginning with the third carbon from the methyl end and is thus designated “22:6 n-3”. Another LCPUFA is EPA which is designated “20:5 n-3”. The biosynthetic pathways for AA (n-6 series) and DHA (n-3 series) from their respective C18 precursors are distinct, but share elongation and desaturation steps and are well understood. Thus, other LCPUFAs are the C18 fatty acids that are precursors in these biosynthetic pathways, for example, linoleic (18:2 n-6) (LA) and gamma-linolenic (18:3 n-6) (GLA) acids in the n-6 pathway, and alpha-linolenic (18:3 n-3) (ALA) and stearidonic (18:4 n-3) (SDA) in the n-3 pathway.

Fatty acids are often found in nature as acyl radicals esterified to alcohols. A glyceride is such an ester of one or more fatty acids with glycerol (1,2,3-propanetriol). If only one position of the glycerol backbone molecule is esterified with a fatty acid, a “monoglyceride” is produced; if two positions are esterified, a “diglyceride” is produced; and if all three positions of the glycerol are esterified with fatty acid a “triglyceride” or “triacylglycerol” is produced. A glyceride is called “simple” if all esterified positions contain the same fatty acid; or “mixed” if different fatty acids are involved. Although a phospholipid can be considered a type of diglyceride, wherein the third position on the glycerol backbone is bonded to a nitrogen containing compound such as choline, serine, ethanolamine, inositol, etc., via a phosphate ester, is not a diglyceride for purposes of this invention. Triglycerides and phospholipids are often classified as long chain or medium chain, according to the fatty acids attached thereto. A “source” of fatty acids may include any of these forms of glycerides from natural or other origins, FFAs, or phospholipids.

Examples of LCPUFAs that are incorporated in the monoglycerides and diglycerides of the invention include AA, EPA, DHA, SDA, LA, ALA, GLA, dihomo-gamma-linolenic (20:3n-6, DHGLA), and the like.

The level of a particular fatty acid in a composition is typically expressed as percent of the total fatty acids. This percentage multiplied by the absolute concentration of total fatty acids in the composition (either as g/L or g/100 kcal) gives the absolute concentration of the fatty acid of interest (in g/L or g/100 kcal, respectively). A listing of the percents of several fatty acids present in a composition is generally referred to as the fatty acid “profile” of the composition. Total fatty acids in triglycerides is estimated as about 95% of total fat to account for the weight of the glycerol backbone, 91% in diglycerides, 81% in monoglycerides, 100% in FFAs and FAMEs, and 74% in phospholipids (to account for the weight of the glycerol backbone and phosphate moiety). Conversion from mg/100 kcal to mg/L is a simple calculation dependant on the caloric density as is known to those skilled in the art.

Compositions

One aspect of the invention relates to an MGDG composition. The MGDG composition contains monoglycerides of a LCPUFA and diglycerides of a LCPUFA. In one embodiment, the MGDG composition contains from about 10 to about 90% by weight of monoglycerides containing monoglycerides of a LCPUFA and from about 10 to about 90% by weight of diglycerides containing diglycerides of a LCPUFA. In another embodiment, the MGDG composition contains from about 20 to about 80% by weight of monoglycerides containing monoglycerides of a LCPUFA and from about 20 to about 80% by weight diglycerides containing diglycerides of a LCPUFA. In yet another embodiment, the MGDG composition contains from about 30 to about 70% by weight of monoglycerides containing monoglycerides of a LCPUFA and from about 30 to about 70% by weight of diglycerides containing diglycerides of a LCPUFA. In still yet another embodiment, the MGDG composition contains about 70% by weight or more of monoglycerides containing monoglycerides of a LCPUFA and about 10% by weight or less of diglycerides containing diglycerides of a LCPUFA.

Based on the combined weights of the monoglycerides and the diglycerides, the MGDG composition typically contains at least about 20% by weight of monoglycerides of a LCPUFA and diglycerides of a LCPUFA. Alternatively, the MGDG composition typically contains at least about 25% by weight of monoglycerides of a LCPUFA and diglycerides of a LCPUFA. Alternatively, the MGDG composition typically contains at least about 30% by weight of monoglycerides of a LCPUFA and diglycerides of a LCPUFA.

Still alternatively, the monoglycerides and diglycerides contain substantial amounts of LCPUFA containing at least about 20 carbon atoms and about 3 or more carbon-carbon double bonds or substantial amounts of LCPUFA containing at least about 22 carbon atoms and about 4 or more carbon-carbon double bonds. For example, in one embodiment, the monoglycerides and diglycerides contain at least about 20% by weight of LCPUFA containing at least about 20 carbon atoms and about 3 or more carbon-carbon double bonds or at least about 10% of LCPUFA containing at least about 22 carbon atoms and about 4 or more carbon-carbon double bonds.

In one embodiment, the MGDG composition contains at least about 15% by weight of monoglycerides and/or diglycerides of one or more of DHA, AA, LA, EPA, ALA, and GLA. In another embodiment, the MGDG composition contains at least about 20% by weight of monoglycerides and/or diglycerides of one or more of DHA, AA, LA, EPA, ALA, and GLA. In yet another embodiment, the MGDG composition contains at least about 25% by weight of monoglycerides and/or diglycerides of one or more of DHA, AA, LA, EPA, ALA, and GLA. In still yet another embodiment, the MGDG composition contains at least about 30% by weight of monoglycerides and/or diglycerides of one or more of DHA, AA, LA, EPA, ALA, and GLA.

In one embodiment, the ratio of the monoglycerides to the diglycerides in the MGDG composition is from about 1:3 to about 10:1. In another embodiment, the ratio of the monoglycerides to the diglycerides in the MGDG composition is from about 1:2 to about 5:1. In yet another embodiment, the ratio of the monoglycerides to the diglycerides in the MGDG composition is from about 1:1.5 to about 3:1. Nevertheless, all ratios are within the scope of the invention.

The monoglycerides include 1-monoglycerides and 2-monoglycerides. Although not a critical feature of the invention, the ratio of 1-monoglycerides to 2-monoglycerides may vary, depending upon the starting materials and/or the reaction conditions employed to make the MGDG composition. In some embodiments, the ratio of 1-monoglycerides to 2-monoglycerides in the MGDG composition is from about 1:4 to about 4:1. In another embodiment, the ratio of 1-monoglycerides to 2-monoglycerides in the MGDG composition is from about 1:2 to about 2:1. For example, when there is a relatively high amount of DHA present as the LCPUFA, the ratio of 1-monoglycerides to 2-monoglycerides in the MGDG composition can be about 4:1, whereas when there is a relatively high amount of AA present as the LCPUFA, the ratio of 1-monoglycerides to 2-monoglycerides in the MGDG composition can be about 2:1. The ratio may also vary depending upon the specific LCPUFA quantified. Nevertheless, all ratios are within the scope of the invention.

The diglycerides include 1,2-diglycerides and 1,3-diglycerides. The diglycerides of a LCPUFA may be simple or mixed. Although not a critical feature of the invention, the ratio of 1,2-diglycerides and 1,3-diglycerides may vary, depending upon the starting materials and/or the reaction conditions employed to make the MGDG composition. In one embodiment, the ratio of 1,2-diglycerides to 1,3-diglycerides is from about 1:4 to about 4:1. In another embodiment, the ratio of 1,2-diglycerides to 1,3-diglycerides is from about 1:2 to about 2:1. For example, when there is a relatively high amount of DHA present as the LCPUFA, the ratio of 1,2-diglycerides to 1,3-diglycerides in the MGDG composition can be about 1:2, whereas when there is a relatively high amount of AA present as the LCPUFA, the ratio of 1,2-diglycerides to 1,3-diglycerides in the MGDG composition can be about 1:2. The ratio may also vary depending upon the specific LCPUFA quantified.

The MGDG composition optionally further contains relatively small amounts of triglycerides. The triglycerides may be simple or mixed. In one embodiment, the MGDG composition contains from about 0 to about 20% by weight of triglycerides containing triglycerides of a LCPUFA. Alternatively, the MGDG composition contains from about 1 to about 10% by weight of triglycerides containing triglycerides of a LCPUFA (about 10% by weight or less of triglycerides). Alternatively, the MGDG composition contains from about 2 to about 6% by weight of triglycerides containing triglycerides of a LCPUFA, including about 5% by weight or less of triglycerides. The triglycerides typically contain at least about 20% by weight of triglycerides of a LCPUFA

The MGDG composition contains substantially more monoglycerides and diglycerides of LCPUFA than triglycerides of LCPUFA, which contributes to one or more of the improved oxidative stability of the MGDG composition, improved digestability, improved shelf life, improved lipase hydrolysis, and improved absorption. In one embodiment, the ratio of the monoglycerides and diglycerides to the triglycerides in the MGDG composition is from about 3:1 to about 100:1. In another embodiment, the ratio of the monoglycerides and diglycerides to the triglycerides in the MGDG composition is at least about 5:1. In yet another embodiment, the ratio of combined monoglycerides and diglycerides to the triglycerides in the MGDG composition is at least about 7:1.

Thus, in one aspect of the invention, the MGDG composition consists essentially of monoglycerides, diglycerides and triglycerides, with monoglycerides and diglycerides being the predominant species. Such compositions may contain only relatively minute quantities (e.g. less than about 2%) of FFA and FAME, as described below, without deviation from the invention.

In yet other aspects, the MGDG composition may further comprise small amounts of FFAs and FAMEs, and in particular, free long chain fatty acids and LCPUFA. In one embodiment, the MGDG composition contains from about 0.1 to about 10% by weight of free long chain fatty acids. In another embodiment, the MGDG composition contains from about 2 to about 5% by weight of free long chain fatty acids.

Generally, the fatty acid profile of the MGDG composition will be the same or nearly the same as the fatty acid profile of the starting materials used to make the MGDG composition. Having a “similar” or “nearly the same” profile in this context means that the level or percent of each fatty acid present in the MGDG composition (as a percent of the total fatty acids) will be approximately the same percent—i.e. within a range of plus or minus about 15%, ideally within about 10% of—as its level or percent in the starting materials.

Process of Making

Generally speaking, MGDG composition is made by hydrolyzing a mixture of triglycerides of LCPUFA and/or phospholipids of LCPUFA to provide FFAs, combining the FFAs with glycerol to esterify the FFAs, and recovering monoglycerides of LCPUFA and/or diglycerides LCPUFA.

Any suitable source of triglycerides of LCPUFA and/or phospholipids of LCPUFA may be employed. Triglyceride and phospholipid sources include marine and fish oils, animal oils, eggs, vegetable oils, microbial oils including bacterial oils, algal oils, fungal sources, blubber, lard, tallow, and the like.

Fish and marine oils are those oils obtained from fish and other marine animals. Examples of marine animals include blue-colored fish, such as the mackerel, sardine, mackerel pike and herring; salmon; cod; plankton, krill and the various shrimp-like copepods. Such oils typically contain DHA in amounts ranging from about 3% to about 20%. Typically, however, fish oils also contain EPA which depresses the production of AA in the body. The addition of a microbial oil containing high levels of AA to fish oil-containing compositions substantially overcomes that problem.

Vegetable oil includes all those oils from plants which contain LCPUFAs. Typically, vegetable oils do not contain large amounts of LCPUFAs (such as at least 20 carbons long). Vegetable oils known to contain LCPUFAs may contain GLA. Examples of vegetable oils include green leafy vegetables such as spinach, broccoli, and purslane; and oilseeds such as soya, sunflower, flax, canola, rapeseed, cotton seeds, black currant seed oil (Ribes nigrum), borage oil (Borago officinalis), and primrose oil (Oenothera biennis). SDA is found in marine oils and plant seeds. Methods for isolating vegetable oils are known to those of skill in the art.

Certain fungi produce LCPUFA-containing oils. Both GLA and AA are found in filamentous fungi. For example, Mucor species produce a GLA-containing oil. AA and GLA can be obtained from an isolated fungus, Mortierella alpina. AA can be obtained from Porphyridium cruentum, a red microalgae.

Numerous microbial sources are available for providing triglycerides of LCPUFA and/or phospholipids of LCPUFA. For example, DHA can be obtained from Crypthecodinium cohnii, EPA can be obtained from Nitzschia alba, and AA can be obtained from species such as Pythium insidiosum or Mortierella alpina. Genetically modified microbial sources may also be employed.

AA can be purified from animal tissues including liver and adrenal gland. Animal sources generally include beef and pork organs.

Egg sources specifically include egg yolks. The egg yolks are generally derived from various avian species such as the hen, turkey, and the like. However, eggs of other animals can be used, for example, that of fish such as salmon eggs as well as eggs of turtles. Additionally, animals fed a diet enriched in specific fatty acids have reportedly produced eggs enriched in said specific fatty acids. Egg yolks can be in different forms such as liquid, frozen, or solid with or without conventional additives such as silica flow agents. Egg yolk solids can be obtained from eggs by various conventional means such as by spray drying egg yolks, freeze drying, etc.

Generally speaking, if necessary, the lipid mixture is separated from the animal or vegetable fat or oil by extraction or leaching with a solvent such as alcohol or hydrocarbon and optionally put through a transesterification process. Examples of solvents for leaching or extracting lipids there can be mentioned lower alkanols having from 1 to about 4 carbon atoms such as methanol, ethanol, isopropanol, and the like; hydrocarbons such as hexane; ethers such as petroleum ether and diethyl ether; lower alkanes under pressure such as those having from 3 to 4 carbon atoms and halogen substituted lower alkanes such as trichloromethane and dichloromethane; ketones such as acetone; as well as mixtures of the foregoing. For example, one or more of the sources of triglycerides of LCPUFA and/or phospholipids of LCPUFA may be mixed with a lower alkanol, e.g., methanol or ethanol, which yields a lipid mixture containing phospholipids, triglycerides and sterols in liquid form, and solid protein material. The solid protein material is easily separated from the lipid mixture by methods known in the art such as filtration or centrifugation.

More specifically, a typical composition of hen's egg yolks as found in Sim, J. S. et al., Egg Uses and Processing Technologies, page 120 (1994) is as follows on a percent by weight basis: (a) 47.5% water, 33.0% lipids, 17.4% protein, 0.20% of carbohydrates (free), 1.1% of inorganic elements; and others of 0.8%; (b) as to lipid composition (from total lipids): triglycerides of 71-73%, cholesterol of 4-6%, phospholipids of 23-25%, lecithin (in phospholipids) of 70-77%, C16-C18 fatty acids 99.5%, saturated fatty acids 44%, monounsaturated fatty acids 44% and polyunsaturated fatty acids of 10.2%.

Egg yolks can be in different forms such as liquid, frozen, or solid with or without conventional additives such as silica flow agents. Egg yolk solids can be obtained from eggs by various conventional means such as by spray drying egg yolks, freeze drying, etc. Egg yolk solids typically have 5% maximum moisture content, a pH of 6.5±0.3, a 56.0 wt % minimum fat content, protein of 30 wt % minimum.

The long chain unsaturated fatty acids such as AA and DHA in egg yolk lipids are found predominantly in the phospholipid fraction. In the methanol solution of the egg yolk lipids, the amount of lipids is typically about 38 wt %; the amount of AA is about 4 wt %; and the amount of DHA is about 1.5 wt % as determined by a relative fatty acid profile. However, the quantity of these lipid components can vary depending on the species of animal, its diet, time of year, etc.

The amount of phosphorus and cholesterol contained in the egg sources is very low. Generally, the quantity of phosphorus can vary from about 0.1 wt % to 0.0001 wt % based on the egg source. Often the quantity of cholesterol is less than 0.5 wt % and particularly less than 0.1 wt % based on the weight of the egg source. The distilled free fatty acids as well as the distilled lower alkyl esters also have the low phosphorus and low cholesterol levels given above for the egg source. It is particularly preferred that the fatty acid and ester products of this invention be essentially free of cholesterol, sterols and phosphorus compounds.

The quantity of organic solvent used for extracting lipids from a lipid source, can vary over a broad range sufficient to dissolve the lipids. In the case of egg yolk solids, such quantity can vary from about 40 ml to over 800 ml of alkanol based on 100 grams (g) of egg yolk solids. Larger quantities of alkanol can be used but such larger quantities serve little useful purpose since it needs to be removed in later steps of the process.

By extracting egg yolk with an alkanol, a phospholipid-rich egg lipid extract is obtained. It is the phospholipids which contain most of the AA and DHA of the egg yolk. The extraction temperature can vary from about 0° C. to the boiling point of the solvent.

The addition of a lower alkanol as used in the extraction of lipids from a lipid source or when simply added to a lipid mixture from which the triglycerides are not separated from the phospholipids before hydrolysis or transesterification causes the formation of two liquid phases when the temperature is maintained between 20° C. and 68° C. The top phase is comprised of phospholipids, sterols, and alcohol, the bottom phase is comprised of triglycerides and sterols. The triglyceride phase is removed by methods known in the art such as decantation. For lipid mixtures such as egg yolks in which the polyunsaturated fatty acids such as AA, DHA and EPA are predominantly bound in the phospholipids rather than the triglycerides, the addition of the alcohol is convenient and inexpensive method of removing the triglycerides and concentrating the polyunsaturated fatty acids in the remaining lipid mixture. The addition of the lower alkanol does not interfere with the subsequent hydrolysis reaction nor the transesterification reaction and can provide the lower alkanol needed for transesterification of the fatty acid portion of the phospholipid. Water can be used to assist in such separation and the quantity of water can vary over a wide range such as that of from about 1 wt % to about 100 wt % based on the source of the lipids, e.g., egg yolk solids.

The processing of LCPUFAs from an egg source is described in U.S. Pat. Nos. 5,883,273 and 6,063,946, incorporated herein by reference. The former describes an alkaline transesterification route via a lower alkyl ester intermediate; the latter describes a hydrolysis route to free fatty acids. Either might be used to extract relevant LCPUFAs from an egg source for use in the present invention.

After removing wastes from and drying the glycerides, the glycerides are optionally subjected to decolorization such as by contact with activated carbon and the solids from such process then removed, e.g., by a filter press to recover the glycerides of AA and DHA together with small quantities of cholesterol and even smaller quantities of phosphorus. Additionally the decolorized glycerides can be deodorized to remove all volatile components such as FFAs, or FAMEs, and residual solvent. Such processing is typical for the production of edible glyceride oils.

Nevertheless, an advantage to employing egg sources such as egg yolk powder as the starting material (source of triglycerides of LCPUFA and/or phospholipids of LCPUFA), is that the extraction described above is unnecessary. That is, egg sources such as egg yolk powder may be hydrolyzed to provide a FFA preparation. That is, egg sources such as egg yolk powder may be hydrolyzed as is, to provide a FFA preparation. After the saponification procedure, the phosphorous in the phospholipids is hydrolyzed from the lipid moiety, and remains in the aqueous layer of the hydrolysis mixture. After acidified of the hydrolysis mixture, the FFAs are extracted by the organic solvents; the phosphorous is thus removed from the FFA preparation. The elimination of phosphorous is advantageous to the resulting FFA preparation, making it more suitable to be used in nutritional product formulation without the possible negative impact of increasing phosphorous content in the final products. Other unwelcome components that are commonly present in the egg yolk are sterol and sterol esters, such as cholesterol and cholesterol esters. Sterol esters present in the phospholipids starting material are hydrolyzed by the saponification procedure. Only free sterols such as cholesterol are left in the hydrolysis mixture. There are numerous methods in the literature for removing sterols from FFA preparations. For example, sterols including cholesterol could be extracted and removed from the FFA after saponification using method described in American Oil Chemists Society Official Method Ca 6b-53. Optionally, sterols and sterol esters can also be separated from FFA using method similar to that described in U.S. Pat. No. 6,063,946 which is hereby incorporated by reference.

Hydrolysis is carried out in any suitable manner to provide FFA from the starting material or source of triglycerides of LCPUFA and/or phospholipids of LCPUFA. The starting material (or extracted starting material) containing phospholipids and/or triglycerides, and in some instances other compounds such as sterols, is hydrolyzed using a suitable amount of base. Base hydrolysis reactions are commonly known as saponification reactions. Examples of bases include alkali, alkaline earth, and ammonium hydroxides, methoxides, ethoxides, carbonates and bicarbonates. Specific examples include lithium, sodium, potassium, magnesium, calcium, ammonium, tetra-alkyl ammonium hydroxides, carbonates and bicarbonates.

Hydrolysis may be carried out in water, or an aqueous system containing water and a polar organic solvent, such as methanol, ethanol, and/or isopropanol. The hydrolysis mixture of solvent, base, and source of triglycerides of LCPUFA and/or phospholipids of LCPUFA is optionally heated to facilitate formation of the FFA preparation. In one embodiment, the hydrolysis mixture is heated to a temperature from about 50° C. to about 140° C. In another embodiment, the hydrolysis mixture is heated to a temperature from about 70° C. to about 110° C. In yet another embodiment, the hydrolysis mixture is heated to temperatures of about 100° C. or less.

The base is added in at least a stoichiometric amount up to about two times the stoichiometric amount based on the equivalents of fatty acid groups contained in the starting material. For example, the amount of base combined with the starting material is from about 1 to about 1.5 times the equivalent of fatty acid groups contained in the starting material.

After base hydrolysis, the pH of the mixture is lowered to about 4 or less using one or more acids. The acids useful for lowering the pH of the mixture must have a pKa lower than the pKa of the FFA. Examples of acids include mineral acids such as hydrochloric acid, sulfuric acid, phosphoric acid, and nitric acid, acetic acid, and the like. In another embodiment, the pH of the mixture is lowered to about 3 or less (including to within the range of about 2 to about 3) using an acid. The acid may be added in dilute or concentrated form.

In some instances, lower alkyl esters (FAMEs) containing from 1 to 5 carbon atoms may form in addition to, or alternatively instead of FFA. FAMEs are processed in the same manner as the FFAs are, as described below. In some instances, FAMEs provide added stability when storing or processing the FFAs.

An organic solvent is added to the acidified hydrolysis mixture to facilitate the formation of a two phase product (aqueous phase and organic phase). The aqueous phase is separated from the organic phase by decantation, extraction, or other suitable techniques. General examples of organic solvents include ethers, esters, alkanols, alkanes, ketones, and the like. Specific examples of organic solvents include dimethyl ether, diethyl ether, dipropyl ether, dibutyl ether, methyl t-butyl ether, methyl phenyl ether and other aliphatic or alkyl aromatic ethers, ethoxy ethanol, butoxy ethanol, ethoxy-2-propanol, propoxy ethanol, butoxy propanol and other glycol ethers, methyl acetate, ethyl acetate, iso-amyl acetate, alkyl carboxylic esters, butanol, iso-butanol, amyl alcohol, cyclohexanol, and other aliphatic alcohols, hexanes, cyclohexane, octanes, tetrahydrofuran, acetone, methyl ethyl ketone, methyl isobutyl ketone, mesityl oxide, methyl amyl ketone, cyclohexanone and other aliphatic ketones, and the like.

Water is added to the acidified alcoholic hydrolysis mixture to dilute the alcohol and facilitate the liberation of FFAs, such that they can be readily extracted by the organic solvents. The addition of the water makes possible the formation of two distinct phases when the organic solvents are added to the hydrolysis mixture. After the separation, the organic phase can be optionally washed using a water or a salt solution. In one embodiment, the organic phase is washed until the water or salt solution is approximately pH neutral (pH of about 7).

Optionally, after base hydrolysis, the pH of the mixture remains to be alkaline, i.e. pH greater than 7. Water is added to the basic alcoholic hydrolysis mixture to dilute the alcohol and facilitate the liberation of FAMEs. Examples of the alcohols include lower alkyl alcohols, such as methanol, ethanol, propanol, iso-propanol, butanol, iso-butanol and the like. The FAMEs are extracted by organic solvents in a similar fashion as described above. The resulted FAMEs can be used in a similar manner as the FFAs; that is, reacted with glycerol for the preparation of mono and diglycerides as disclosed in this invention.

A FFA preparation is obtained from the organic phase using rotoevaporation, distillation, or other suitable technique. In one embodiment, rotoevaporation under a vacuum or vacuum distillation is employed. The FFA preparation is substantially and preferably completely free of unmodified triglycerides or phospholipids from the starting materials.

In some instances it is desirable to increase the ratio of the unsaturated FFA in relation to the saturated FFA. This can be accomplished by various fractionation techniques such as solvent fractionation, solid fractionation, molecular fraction distillation, winterization, etc. Such fractionation can rely on the difference in the melting, boiling, or solidification temperatures of the saturated or monounsaturated FFA and esters thereof in relation to the polyunsaturated FFA and esters thereof. The fractionation can be applied to the FFAs and their esters before esterification.

The FFA preparation is combined with a suitable excess amount glycerol (1,2,3-propanetriol) and heated at a suitable temperature for a suitable time to provide a MGDG composition. In one embodiment, the weight ratio of LCPUFA in the FFA preparation to glycerol is from about 1:5 to about 1:100. In another embodiment, the weight ratio of LCPUFA to glycerol is from about 1:30 to about 1:80. In yet another embodiment, the weight ratio of LCPUFA to glycerol is from about 1:40 to about 1:70.

In one embodiment, the FFA preparation and glycerol esterification mixture is heated at a temperature from about 70° C. to about 150° C. for a time from about 1 to about 24 hours. In another embodiment, the FFA preparation and glycerol esterification mixture is heated at a temperature from about 90° C. to about 140° C. for a time from about 2 to about 15 hours. In yet another embodiment, the FFA preparation and glycerol esterification mixture is heated at a temperature from about 110° C. to about 130° C. (about 130° C. or less) for a time from about 4 to about 10 hours.

Optionally, the FFA preparation is combined with glycerol in the presence of an antioxidant. In some instances, the antioxidant prevents undesirable oxidation of the LCPUFA during esterification. Examples of antioxidants include phenolic antioxidants such as tocopherols, butylated hydroxy toluene, butylated hydroxy anisole, beta carotene, ascorbyl palmitate, and the like. In one embodiment, from about 0.1% to about 5% by weight of antioxidant per FFA preparation is added to the esterification mixture. In another embodiment, from about 0.25% to about 2% by weight of antioxidant per FFA preparation is added to the esterification mixture.

Optionally, the FFA preparation is combined with glycerol in the presence of an emulsifier. In some instances, the emulsifier facilitates interaction between the LCPUFA and glycerol. General examples of emusifiers include monoalkylglycerides, soy mono, diglycerides, and soy lecithin. A specific example of an emulsifier includes distilled monoglyceride from soybean oil such as that available from Danisco Ingredient USA, Inc. In one embodiment, from about 0.1% to about 5% by weight of emulsifier per FFA preparation is added to the esterification mixture. In another embodiment, from about 1% to about 3% by weight of emulsifier per FFA preparation is added to the esterification mixture.

Optionally, the FFA preparation is combined with glycerol in the presence of an esterification catalyst. In some instances, the esterification catalyst improves the efficiency of the esterification reaction. Examples of esterification catalysts include organic sulfonic acids such as p-toluene sulfonic acid. In one embodiment, about 1% to about 15% by weight of esterification catalyst per FFA preparation is added to the esterification mixture. In another embodiment, about 6% to about 12% by weight of esterification catalyst per FFA preparation is added to the esterification mixture.

The monoglycerides and diglycerides are separated from the esterification reaction by extraction, decantation, or other suitable technique. The organic solvents mentioned above in connection with separating the aqueous phase from the organic phase from the hydrolysis mixture can be employed to isolate and/or recover the MGDG composition.

After separation of the MGDG composition from the esterification reaction, optionally purification schemes such as deodorization, refining, bleaching, and decoloration may be employed using standard procedures known in the art of processing edible oils. Such optional processes remove volatile components such as FFA, lower alkyl esters, residual solvents, and the like.

The MGDG composition produced in accordance with this invention can be further processed to concentrate the levels of LCPUFA such as AA and DHA containing glycerides. Such additional processing includes freeze fractionation, super critical extractions and enzymatic transesterification.

Nutritional Products

The MGDG composition can be incorporated into liquid, slurry, and powder nutritional products. Another aspect of the invention thus relates to nutritional formulations and nutritional supplements containing the MGDG composition.

Briefly, nutritional formulations and nutritional supplements are formed by suitably combining and processing component preparations. Component preparations of the nutritional formulations (such as one or more of a protein component, carbohydrate component, and fat component), typically in the form of slurries, are prepared, blended together, heat treated, standardized, spray dried (if applicable), packaged and sterilized (if applicable). A fat component preparation or nutritional supplement may contain or be constituted by the MGDG composition.

For example, a liquid nutritional formulation is made as follows. A carbohydrate preparation or slurry is prepared by first heating water to an elevated temperature with agitation. A carbohydrate source, such as one or more of lactose, corn syrup solids, sucrose and/or maltodextrin is dissolved in the water, thereby forming a carbohydrate solution. A source of dietary fiber, such as soy polysaccharide, may also be added. The completed carbohydrate preparation or slurry is held under agitation at elevated temperature until it is blended with the other component preparations or slurries, generally for no longer than about twelve hours.

A mineral preparation or slurry is made by adding minerals to water. Examples of minerals include sodium citrate, sodium chloride, potassium citrate, potassium chloride, magnesium chloride, tricalcium phosphate, calcium carbonate, potassium iodide, trace mineral premix, and the like. Alternatively, minerals can be added to the carbohydrate preparation, protein preparation, fat preparation, or MGDG composition.

A protein in water slurry is prepared by first heating water to an appropriate elevated temperature with agitation. The protein source is then added to the water with agitation. Typically this protein source is intact or hydrolyzed milk proteins (e.g. whey, casein), intact or hydrolyzed vegetable proteins (e.g. soy), free amino acids and mixtures thereof. In general, any known source of amino nitrogen can be used in this invention. The completed protein slurry is held under agitation at elevated temperature until it is blended with the other preparations or slurries, typically for a period no longer than about two hours. As an alternative, some protein may be mixed in a protein-in-fat emulsion rather than protein-in-water.

A fat preparation is prepared by providing at least the MGDG composition. The fat preparation can optionally further contain one or more of soy, coconut, palm olein, high oleic safflower and sunflower oils, medium chain triglycerides, and long chain triglycerides.

Emulsifiers, such as diacetyl tartaric acid esters of mono, diglycerides, soy mono, diglycerides, and soy lecithin may be added to a component preparation or to the nutritional formulation, but are typically added to the fat blend. Any or all of the oil-soluble vitamins A, D, E (natural R,R,R form or synthetic) and K may be added individually or as part of a premix to a component preparation or to the nutritional formulation. Beta carotene, which can function as an in vivo antioxidant, may also be added to a component preparation or to the nutritional formulation, as may a stabilizer such as carrageenan.

The protein preparation and carbohydrate preparation are blended together with agitation and the resultant blended slurry is maintained at an elevated temperature. After a brief delay (e.g. for about a few minutes), the fat blend is added to the blended slurry from the preceding step with agitation, where the MGDG composition is a component of the fat blend or constitutes the fat blend.

After sufficient agitation to thoroughly combine all constituents, the pH of the completed blend is adjusted to the desired range. The blended slurry is then subjected to at least one of deaeration, ultra-high temperature heat treatment, emulsification and homogenization, then is cooled to refrigerated temperature. After forming the nutritional formulation, appropriate analytical testing for quality control is optionally conducted. Based on the analytical results of the quality control tests, and appropriate amount of water is added to the batch with agitation for dilution.

A vitamin solution, containing water soluble vitamins and trace minerals (such as including sodium selenate), may be prepared and optionally added to the processed slurry blend with agitation. Alternatively, the vitamin solution can be added to either the protein component or carbohydrate component before blending.

The pH of the final product may be adjusted again to achieve optimal product stability. The completed product is then filled into the appropriate metal, glass or plastic containers and subjected to terminal sterilization using conventional technology. Alternatively, the liquid product can be sterilized aseptically and filled into plastic containers.

A nutritional formulation in powder form can also be made. A protein preparation, carbohydrate preparation and mineral preparation are prepared as described above for the liquid nutritional formulation.

A fat preparation containing the MGDG composition is prepared as described above for liquid product manufacture with the following exceptions: 1) Emulsifiers and stabilizers typically are not added to powder, and 2) In addition to the beta carotene, other antioxidants, such as mixed tocopherols and ascorbyl palmitate, can be added to help maintain the oxidative quality of the product during any subsequent spray drying process.

The protein and carbohydrate preparations are blended together in a similar manner as described for liquid product manufacture. After pH adjustment of the completed blend, the MGDG composition is added via the fat blend to the blended slurry with agitation. Desirably, fat blend containing the MGDG composition is slowly metered into the product as the blend passes through a conduit at a constant rate just prior to homogenization (in-line blending).

After at least one of deaeration, ultra-high temperature heat treatment, emulsification and homogenization, the processed blend may be evaporated to increase the solids level of the blend to facilitate more efficient spray drying. The blend then passes through a preheater and a high pressure pump and is spray dried using conventional spray drying technology. The spray dried powder may be agglomerated, and then is packaged into metal or plastic cans or foil/laminate pouches under vacuum, nitrogen, or other inert environment.

Nutritional formulations contain protein, carbohydrate, and fats, where the fats are supplied, at least in part, by the MGDG composition. In one embodiment, liquid nutritional formulations contain from about 5 to about 30 g/l protein, from about 20 to about 150 g/l carbohydrate, and from about 5 to about 60 g/l fats. In another embodiment, liquid nutritional formulations contain from about 10 to about 25 g/l protein, from about 50 to about 120 g/l carbohydrate, and from about 10 to about 50 g/l fats. In such nutritional formulations, from about 2% to about 60% by weight of the fats are LCPUFA containing compounds. Alternatively, from about 2% to 100% of the fat blend may be supplied by the MGDG Composition, more typically from about 2% to about 50% in infant nutritionals and from about 5% up to about 90% in adult nutritional formulas.

Nutritional formulations may be administered to infants, children, or adults. Infant formula refers to specifically nutritional formulations that meet the standards and criteria of the Infant Formula Act, (21 USC §350(a) et. seq.) and are intended to replace or supplement human breast milk. Although such formulas are available in at least three distinct forms (powder, liquid concentrate and liquid ready-to-feed (“RTF”), it is conventional to speak of the nutrient concentrations on an “as fed” basis and therefore the RTF is often described, it being understood that the other forms reconstitute or dilute according to manufacturer's directions to essentially the same composition and that one skilled in the art can calculate the relevant composition for concentrated or powder forms.

There are a number of benefits, advantages, and uses for the MGDG composition and nutritional formulations containing the MGDG composition, particularly when comparing the MGDG composition to a corresponding triglyceride composition.

Methods of Use

Another aspect of the invention involves using the MGDG composition to deliver LCPUFAs to an organism, such as a mammal or human. As a result, the present invention further includes methods of treating various conditions, symptoms, diseases, and/or disorders where delivery of LCPUFAs is in order.

Specifically, the present invention includes methods of administering an effective amount of the MGDG composition to an organism to treat conditions, symptoms, diseases, and/or disorders associated with one or more of restenosis after angioplasty, symptoms of inflammation and rheumatoid arthritis, treatments for asthma and psoriasis, improving calcium metabolism, treatment or prevention of osteoporosis and of kidney or urinary tract stones, treatment of cancer, increasing a cancer cell's susceptibility to chemotherapeutic agents, treating cachexia associated with cancer, treating diabetes, treating eczema, reducing increases in blood pressure associated with stress, improving performance on arithmetic tests, inhibiting platelet aggregation, lowering cholesterol levels, inhibiting proliferation of vessel wall smooth muscle and fibrous tissue, reducing or preventing gastro-intestinal bleeding and other side effects caused by non-steroidal anti-inflammatory drugs, preventing or treating endometriosis and premenstrual syndrome, treating myalgic encephalomyelitis and chronic fatigue after viral infections, improving performance of eicosenoids that exhibit regulatory effects on lipoprotein metabolism, blood rheology, vascular tone, leucocyte function and platelet activation, improving blood clotting, improving inflammatory and anti-inflammatory response, improving cholesterol absorption, improving bronchial function, lowering hypertension, improving visual acuity and brain development in infants, and improving gastric secretions.

Comparing the MGDG composition to a corresponding triglyceride composition (substantially the same fatty acids bonded to the glycerine backbone), the MGDG composition exhibits increased oxidative stability. Nutritional formulations containing the MGDG composition have LCPUFAs that have increased oxidative stability compared to nutritional formulations containing LCPUFAs in triglyceride form. This means that nutritional formulations containing the MGDG composition may have a longer shelf life than nutritional formulations containing LCPUFAs in triglyceride form.

Since LCPUFAs are absorbed in cells in monoglyceride form, increased absorption rates may be realized for LCPUFAs when delivered in the MGDG composition compared to LCPUFAs delivered in triglyceride form. Lipases are responsible for converting triglycerides to mono and diglycerides. The MGDG composition is thus appropriate for delivering LCPUFA to organisms including humans that have lipase deficiencies.

The MGDG composition, in addition to providing beneficial nutritional and health characteristics, may have emulsification properties. In nutritional formulations containing proteins, carbohydrates, and fats including the MGDG composition, in some instances it is not necessary to provide an emulsifier.

Since the MGDG composition is the product of a synthetic pathway, rather than a modified or processed natural LCPUFA compositions, the MGDG composition is free of many compounds such as sterols and sterol fatty acids often present in modified or processed natural LCPUFA compositions. Therefore, another aspect of the present invention relates to MGDG compositions that are substantially free of sterols, phospholipids, cholesterol, cholesterol esters, proteins, and the like, in addition to having relatively small amounts of triglycerides and FFA.

### EXAMPLES

The following examples illustrate the present invention. Unless otherwise indicated in the following examples and elsewhere in the specification and claims, all parts and percentages are by weight, all temperatures are in degrees Celsius, and pressure is at or near atmospheric pressure.

### Example 1

### Preparation of Mono and Diglyceride According to Invention

1.1) Preparation of Free Fatty Acids of High DHA Oil (FFA-DHA Preparation)

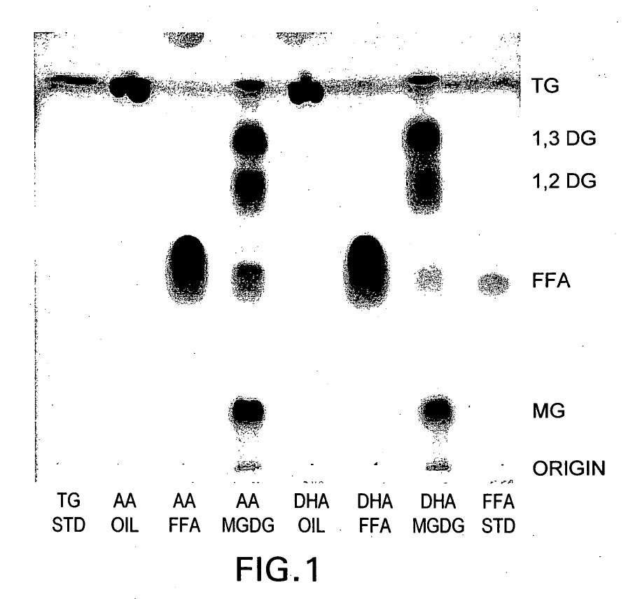

Commercially available fish oil, High DHA Tuna, is purchased from Mochida International Co. Ltd, Nippon Suisan Kaisha Ltd. The typical fatty acid profile is shown in Table 1. A total of 120 g of the High DHA tuna triglyceride oil (TG-DHA) is dispensed into 48 screw cap tubes. Approximately 2.5 g of the TG-DHA oil is pipetted into each tube. Thirty ml of 0.5N ethanolic KOH is added into each tube. The tubes are capped tight, mixed and heated at 100° C. in a boiling water bath for one hour. The tubes are then cooled to room temperature. The resulting FFA of the fish oil is extracted as follows: The content of 8 tubes is transferred into a 1-liter separatory funnel. Lab water 240 ml is added and mixed well. Concentrated hydrochloric acid 12-14 ml is added to acidify the mixture. The pH is tested with pH paper to make sure that the pH is between 2-3. The liberated FFA is extracted twice with 240 ml of ethyl ether (EE). The combined EE extracts are washed thrice with 240 ml of lab water. The pH of the last water wash is tested with pH paper. If not neutral, continue washing. The FFA-DHA in all 48 tubes is extracted in the same manner. The EE extracts are combined and evaporated using rotoevaporation under vacuum at 50-60° C. The FFA-DHA extracts are dissolved in approximately 300 ml of EE and dried over anhydrous sodium sulfate overnight. The dried EE extract is decanted into a preweighed 1 liter round bottom flask and rotoevaporated to remove the solvent. The total FFA-DHA preparation is weighed and the yield calculated. A total of 109 g, or 96% of theoretical yield, of FFA is recovered. The FFA-DHA prepared is completely free of the triglyceride from the starting oil. See FIG. 1 and Table 2. The fatty acid profile of the free fatty acid preparation is similar (generally within about 15% of each value) to those of the starting oil. See Table 1. Larger batch size of FFA can be prepared in a similar manner using larger volume container in industrial manufacturing facilities.

TABLE 1
Fatty Acid Profile of High DHA Tuna Triglyceride Oil
(TG-DHA), Free Fatty Acid (FFA-DHA) and Mono-
Diglycerides (MGDG-DHA) Preparations

| | TG-DHA | FFA-DHA | MGDG-DHA |
|---|---|---|---|
| Fatty Acid | Starting Oil | Preparation | Preparation |
| C14:0 | 3.36 | 3.50 | 3.46 |
| C15:0 | 0.80 | 0.83 | 0.83 |
| C16:0 | 18.65 | 19.48 | 19.80 |
| C16:1n−7 | 4.61 | 4.80 | 4.50 |
| C16:2 | 0.70 | 0.73 | 0.70 |
| C17:0 | 0.98 | 1.03 | 1.10 |
| C16:3 | 0.78 | 0.82 | 0.99 |
| C16:4 | 0.11 | 0.12 | 0.36 |
| c18:0 | 4.69 | 4.92 | 7.61 |
| C18:1n−9 | 16.96 | 17.76 | 17.28 |
| C18:1n−7 | 2.51 | 2.63 | 2.66 |
| C18:2n−6 | 1.64 | 1.52 | 1.58 |
| C18:3n−3 | 0.64 | 0.71 | 0.67 |
| C18:4n−3 | 1.35 | 1.36 | 1.24 |
| C20:1n−9 | 2.25 | 2.35 | 2.38 |
| C20:4n−6 | 1.80 | 1.84 | 1.76 |
| C20:4n−3 | 0.59 | 0.59 | 0.62 |
| C20:5n−3 | 7.00 | 6.56 | 5.81 |
| C22:1n−9 | 2.15 | 2.41 | 2.46 |
| c22:5n−6 | 1.06 | 0.99 | 0.99 |
| C22:5n−3 | 1.48 | 1.42 | 1.33 |
| C22:6n−3 | 23.22 | 20.73 | 18.69 |
| C24:1n−9 | 0.77 | 0.81 | 0.81 |
| Others | 1.89 | 2.06 | 2.37 |
| Total | 100.00 | 100.00 | 100.00 |

TABLE 2
Neutral Lipid Classes Composition of High DHA Tuna
Triglycerides Oil (TG-DHA), Free Fatty Acid (FFA-DHA)
and Mono- Diglycerides (MGDG-DHA)
Preparations

| | TG-DHA | FFA-DHA | MGDG-DHA |
|---|---|---|---|
| | Starting Oil | Preparation | Preparation |
| Triglyceride | 100 | ND | 6.44 |
| Diglyceride | ND | ND | 50.47 |
| Monoglyceride | ND | ND | 39.11 |
| Free Fatty Acid | ND | 100 | 3.07 |

ND = Not Detected

1.2) Preparation of High DHA Mono and Diglycerides (MGDG-DHA Preparation)

The FFA-DHA preparation, 108 g, is dissolved in 1 liter of methylene chloride. To this solution, 0.5 g of mixed tocopherols (Tenox GT-2, Eastman Kodak or equivalent) in 10 ml of methylene chloride and 3 g of distilled monoglyceride from hardened soybean oil (Danisco Ingredient USA, Inc. or equivalent) in 10 ml of methylene chloride are added and mixed well. Three ml aliquot of this solution, containing approximately 0.3 g of FFA-DHA is pipetted into a 50 ml screw cap glass tube and the content is evaporated to dryness with nitrogen. To each tube, approximately 15-30 mg of p-toluene sulfonic acid, 14 ml (17 g) of glycerin (1,2,3-propanetriol, purchased from Sigma Aldrich Chemical Co.) and a small stir bar are added. The sample is flushed with nitrogen and capped tight. A total of 330 tubes are needed for each 100 g of FFA preparation. In order to process the samples within a 12-14 hour workday, the tubes are separated into three batches, approximately 110-120 tubes per batch. Each batch is processed in the same manner as described in the following. The tubes are heated at 120° C. for 5-8 hours with constant stirring using a Pierce Reacti-Therm Heating/Stirring Module (#18935) purchased from Pierce Company (P.O. Box 117 Rockford, Ill. 61105). A thermometer is used to monitor the heating temperature. After heating for 5 hours, a small amount, 0.1-0.2 ml, of sample is taken from one of the sample tube periodically and test for the completion of the reaction using thin layer chromatography. The reaction is determined to be complete by the disappearance of the FFA and the appearance of mono- di- and triglycerides. See FIG. 1. The goal of the reaction is to obtain the maximum yield of mono and diglycerides and the minimum amount of unreacted FFA is left. In addition, the yield of triglyceride is also kept at a minimum. When the reaction is determined to be complete, as shown in FIG. 1, the heat is turned off and the tubes are cooled to room temperature. Lab water, 10 ml, is added to each tube. The content of 8 tubes is transferred to a 1-liter separatory funnel, and the tubes are rinsed with additional 10 ml of lab water. The reaction mixture is extracted twice with 250 ml of EE. The EE extracts are combined, washed once with 250 ml of lab water. All the samples are processed in the same manner and the EE extracts are combined. The majority of the solvent is removed by rotoevaporation and the finish product is dissolved in approximately 350-500 ml of EE. The EE extract is dried over sodium sulfate overnight. Mixed tocopherols 0.5 g are added and the solvent is evaporated by rotoevaporation under vacuum and flushed with nitrogen. The products of the reaction are weighed and the yield is calculated. The weight of the products is 113 g, or 95% of theoretical yield. The product contains of 39.11% of monoglyceride, 50.47% of diglyceride, 6.44% of triglyceride, and 3.07% of FFA. See FIG. 1 and Table 2. The fatty acid profile of the MGDG-DHA Preparation is shown in Table 1. Due to the addition of 3% of distilled monoglyceride in the reaction mixture, the stearic acid, (C18:0) being the major fatty acid in the distilled monoglyceride of hardened soy oil, is increased by approximately 2.7% in the finish product, while the DHA (C22:6 n-3) is reduced concomitantly by approximately 2%. The overall fatty acid profile of the MGDG-DHA Preparation is very similar (i.e. each fatty acid is within a range of plus or minus about 15% of the starting value) to the FFA-DHA Preparation and the TG-DHA starting material. See Table 1.

1.3) Oxidative Stability of the High DHA Mono and Diglycerides Preparation (MGDG-DHA Preparation)

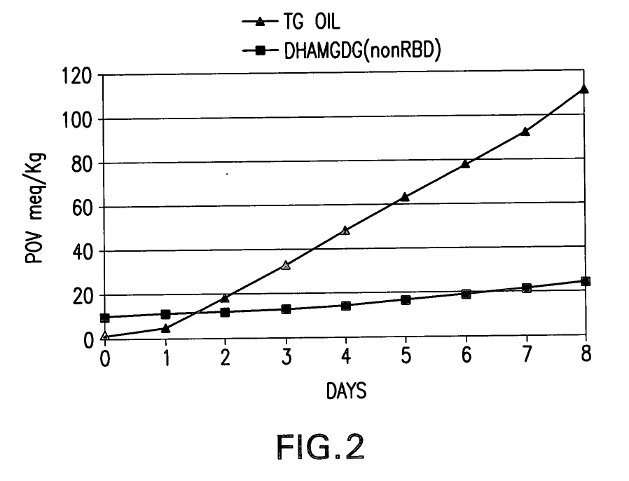

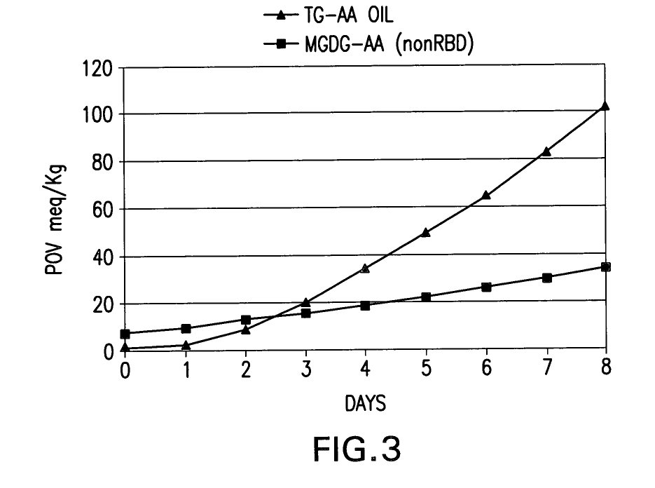

Approximately 50 g of each of the High DHA Tuna triglyceride oil (TG-DHA) and the MGDG-DHA Preparation are placed in a 150 ml beaker. The samples are heated in a sand bath at 40° C. for 8 days in an open air environment. The TG-DHA contains about 0.5-0.7% of tocopherols as antioxidant, whereas the MGDG-DHA preparation contains 0.5% tocopherols as antioxidant. The temperature of the sand bath is controlled using a Thermolyne DRI-BATH (Model DB-28125, Barnstead/Thermolyne, 2555 Kerper Boulevard, Dubuque, Iowa, 52001). A 1-2 g sample is removed each day and the peroxide values are determined using a Metrohm Titrator (Metrohm 785 DMV Titrino, Brinkmann Instruments, Inc. One Cantilague Road, Westbury, N.Y. 11590-0207). The samples are dissolved in chloroform:acetic acid 2:3 (v:v) and titrated with standardized sodium thiosulfate. The concentration of the sodium thiosulfate used is 0.002N for Day 0-3, 0.05N for Day 4-7, and 0.1N for Day 8. The peroxide value of each sample is reported as milliequivalent (meq) of peroxide per kilogram of sample. The results are tabulated in Table 3 and and plotted in FIG. 2. The LCPUFA in the High DHA Tuna triglyceride oil (TG-DHA) is oxidized rapidly under this experimental condition while the LCPUFA in the MGDG-DHA Preparation maintained very stable. The MGDG preparation is a crude preparation from the esterification reaction described in Section 2 above and is not refined, bleached and/or deodorized (RBD). The TG-DHA oil is a commercial edible grade oil, which is refined, bleached and deodorized. Thus the starting peroxide value of the MGDG-DHA Preparation is much higher than the RBD triglyceride oil (TG-DHA) on Day 0. Despite of this disadvantage of the MGDG-DHA Preparation, the oxidative stability of the LCPUFA in the MGDG-DHA Preparation is superior to that of the LCPUFA in the triglyceride oil. At day 2, the peroxide value of the LCPUFA in the triglyceride oil is 160% of those in the MGDG preparation. The trend continues, and on Day 8, the peroxide value of the LCPUFA in the triglyceride oil is 460% of that in the MGDG preparation. This significant improvement of the oxidative stability of the LCPUFA in the MGDG preparation creates a novel product and method that can be used in nutritional product applications. The LCPUFA-MGDG preparation presents the LCPUFA in a form that they are extremely stable under elevated temperature and open air environment from the oxidation degradation. The derivatization of LCPUFA triglycerides to mono and diglycerides provides a novel method to preserve the nutritional values of these heat and oxygen labile fatty acids, prolong the shelf-life of nutritional products containing these valuable fatty acids, and greatly reduced the potential detrimental harmful effects caused by oxidation damage.

TABLE 3
Determination of Peroxide Value (meq/Kg) of High DHA TG Oil and
MGDG-DHA Preparation at 40° C. and Open Air

| Day | TG-DHA Oil | MGDG-DHA (nonRBD) |
|---|---|---|
| 0 | 1.72 | 10.00 |
| 1 | 5.38 | 11.49 |
| 2 | 18.47 | 11.82 |
| 3 | 33.00 | 12.83 |
| 4 | 48.72 | 14.41 |
| 5 | 63.48 | 16.78 |
| 6 | 78.15 | 19.01 |
| 7 | 92.42 | 21.70 |
| 8 | 111.86 | 24.30 |

1.4) Improved Emulsification

Mono and diglycerides are amphiphilic lipids, having both hydrophilic and lipophilic properties which enable them to act as surface active agents and facilitate the interface of oil and water. Mono and diglyceride of soybean oil can play important roles in stabilizing the matrices containing oil in water, such as those found in infant formula, nutritional supplements, and medical nutritional products. The MGDG-LCPUFA Preparation as shown in this invention containing approximately 90% of amphiphilic lipid molecules, Table 2, such that they not only can provide an oxidative stable molecule to supply the LCPUFA in the nutritional products, but also could facilitate a more stable matrix for the finished products. The improvement of the emulsion can improve the physical stability, and give rise to longer shelf-life of the products.

1.5) Improved Absorption

Approximately 90% of dietary fat is consumed as triglycerides. The triglycerides in the diet are first hydrolyzed by lingual and pancreatic lipases to fatty acid and mono and diacylglycerides before absorption. The pancreatic lipase is a 1,3-specific lipase, that is it cleaves fatty acyl groups from the positions 1 and 3 of triglycerides at rates many times greater than that for the 2-acyl groups. The majority of fatty acids esterifies at the 2 position of the glycerol backbone are absorbed as 2-monoglycerides (2-MG). The 2-MG is the major substrate for the re-synthesis of triglycerides which are secreted in the chylomicrons in lymph. The presentation of LCPUFA as mono and diglycerides may reduce the needs of the hydrolysis action by the lipases. For individuals that are deficient or compromised in the lipase activities, fatty acids fed in the mono and diglycerides forms can be absorbed easier and in a faster rate since no hydrolysis by the lipases is required.

It is reported that the pancreatic lipase activity is not efficient for n-3 fatty acids, such as docosahexaenoic acid (DHA, 22:6 n-3) and eicosapentaenoic acid (EPA, 20:5 n-3). It is also reported that fatty acids located in the sn-2 position of the glycerol are better absorbed by the lymphatic system. The MGDG-DHA Preparation from this invention is analyzed to determine the positional distribution of the DHA on the glycerol backbone. The fatty acid profiles in the 1-MG and the 2-MG of the MGDG-DHA Preparation are analyzed and the results are displayed in Table 4. The DHA is found to be predominantly esterified in the 2-MG molecules. The DHA represented 53% of the fatty acids in the 2-MG molecules, and only represented 9% of the fatty acids in the 1-MG molecules. Although the 1-MG is more abundant than the 2-MG in the MGDG-DHA Preparation, as shown in Table 5, the overall percentage of total DHA in the 2-MG is 23.45% whereas only 15.56% of total DHA is in the 1-MG molecules. See Table 6. The DHA in the 2-MG is 150% of that in the 1-MG. Based on the positional distribution of the DHA in the MGDG-DHA Preparation created according to this invention, the absorption of the DHA present in this preparation can be superior to those present in the triglyceride forms.

TABLE 4
Relative Fatty Acid Profile of 1-MG AND 2-MG of Preparations
MGDG-DHA

| | DHA 1-MG | DHA 2-MG |
|---|---|---|
| C14:0 | 4.9 | 1.2 |
| C15:0 | 1.1 | — |
| C16:0 | 25.6 | 5.6 |
| C16:1 | 6.1 | — |
| C16:2 | 0.8 | — |
| C17:0 | 1.2 | — |
| C16:3 | 1.0 | — |
| C18:0 | 9.2 | 2.4 |
| C18:1n−9 | 21.3 | 6.0 |
| C18:1n−7 | 3.4 | — |
| C18:2n−6 | 1.8 | 2.2 |
| C18:3n−3 | 0.7 | — |
| C18:4n−3 | 1.3 | — |
| C20:1n−9 | 2.5 | 1.4 |
| C20:4n−6 | 1.3 | 5.5 |
| C20:5n−3 | 5.3 | 10.6 |
| C22:1n−9 | 0.4 | 3.4 |
| C22:5n−6 | 0.4 | 2.7 |
| C22:5n−3 | 0.8 | 3.1 |
| C24:0 | — | 1.3 |
| C22:6n−3 | 9.0 | 53.0 |
| C24:1n−9 | 0.5 | 1.7 |
| Others | 1.3 | 0.0 |
| Total | 100.0 | 100.0 |

TABLE 5
Positional Distribution of Mono- and Diglycerides Preparation of High
DHA Oil

| | MGDG-DHA |
|---|---|
| | % |
| Total MG | 39.11 |
| 1-MG(Rel to Total MG) | 79.69 |
| 2-MG(Rel to Total MG) | 20.31 |
| MG Ratio (1-MG/2-MG) | 3.92 |
| Total DG | 50.47 |
| 1,3-DG(Rel to Total DG) | 65.73 |
| 1,2-DG(Rel to total DG) | 34.27 |
| DG Ratio (1,3-/1,2-) | 1.92 |
| DG/MG | 1.29 |

TABLE 6
Distribution of DHA in Neutral Lipid Classes of MGDG-DHA

| Lipid Classes | % |
|---|---|
| 1-MG | 15.56 |
| 2-MG | 23.45 |
| FFA | 2.26 |
| 1,2-DG | 14.64 |
| 1,3-DG | 37.77 |
| TG | 6.33 |
| Total | 100.00 |

### Example 2

### Preparation of Mono and Diglyceride According to the Invention

2.1) Preparation of Free Fatty Acids of High Arachidonic Acid (FFA-AA Preparation)

High Arachidonic Acid triglyceride oil (TG-AA) is obtained from a commercial supplier. The typical fatty acid profile of this oil is shown in Table 7. The procedure for the preparation of FFA from TG-AA is similar to that described in Section 1.1) above. The total yield of the FFA from 120 g of TG-AA oil is 105 g, 92% of theoretical yield. The fatty acid profile of the FFA-AA preparation is displayed in Table 8, and is similar to that of the triglyceride oil starting material, TG-AA. The FFA-AA preparation contains exclusively FFA, and no trace of triglyceride from the starting oil is found in the extract. See FIG. 1 and Table 8.

TABLE 7
Fatty Acid Profile of High AA Triglyceride Oil (TG-AA),
Free Fatty Acid (FFA-AA) and Mono-Diglycerides
(MGDG-AA) Preparations

| | TG-AA | FFA-AA | MGDG-AA |
|---|---|---|---|
| Fatty Acid | Starting Oil | Preparation | Preparation |
| C16:0 | 11.49 | 11.74 | 12.18 |
| C18:0 | 7.52 | 7.71 | 10.37 |
| C18:1n−9 | 7.45 | 7.65 | 7.68 |
| C18:2n−6 | 9.91 | 9.99 | 9.50 |
| C18:3n−6 | 2.49 | 2.47 | 2.24 |
| C18:3n−3 | 0.51 | 0.50 | 0.46 |
| C20:0 | 0.88 | 0.91 | 0.95 |
| C20:1n−9 | 0.49 | 0.52 | 0.52 |
| C20:2n−6 | 0.72 | 0.73 | 0.75 |
| C20:3n−6 | 3.51 | 3.45 | 3.35 |
| C20:4n−6 | 40.51 | 38.68 | 36.32 |
| C22:0 | 3.37 | 3.74 | 3.71 |
| C22:1n−9 | 0.12 | 0.28 | 0.20 |
| C22:4n−6 | 0.60 | 0.58 | 0.56 |
| C24:0 | 8.68 | 8.97 | 9.16 |
| Others | 1.77 | 2.08 | 2.04 |
| Total | 100.00 | 100.00 | 100.00 |

TABLE 8
Neutral Lipid Classes Composition of Triglyceride Oil (TG-AA), Free
Fatty Acid (FFA-AA) and Mono- Diglycerides
(MGDG-AA) Preparations

| | TG-AA | FFA-AA | MGDG-AA |
|---|---|---|---|
| | Starting Oil | Preparation | Preparation |
| Triglyceride | 100 | ND | 5.78 |
| Diglyceride | ND | ND | 46.13 |
| Monoglyceride | ND | ND | 37.89 |
| Free Fatty | ND | 100 | 9.02 |
| Acid | | | |

2.2) Preparation of High AA Mono and Diglycerides (High AA-MGDG Preparation)

The FFA-AA Preparation, 105 g, is used to prepare the mono and diglycerides using the procedure described in Section 1.2) above. A total of 106 g of MGDG-AA Preparation are obtained from this procedure. The yield is calculated to be 92% of theoretical. The fatty acid profile of the MGDG-AA Preparation is similar to that of FFA-AA Preparation and the TG-AA starting oil. See Table 7. The preparation contains 37.89% of monoglyceride, 46.13% of diglyceride, 5.78% of triglyceride, and 9.02% of free fatty acid. See FIG. 1 and Table 8.

2.3) Oxidative Stability of the High AA Mono and Diglycerides (MGDG-AA Preparation)

Approximately 50 g of each of the triglyceride oil (TG-AA) and the MGDG-AA Preparation are placed in a 150 ml beaker. The samples are heated at 40° C. for 8 days. The peroxide value of these two samples is determined as described in Section 1.3) above. Similar results are observed with the MGDG-AA and the MGDG-DHA Preparations. The LCPUFA in the TG-AA triglyceride oil is oxidized rapidly under the experimental condition while the LCPUFA in the MGDG-AA Preparation maintain very stable. The MGDG-AA Preparation is not refined, bleached and deodorized and the initial peroxide value is higher than that of the TG-AA oil. In spite of this disadvantage, at day 3, the peroxide value of the LCPUFA in the TG-AA oil is 127% of that in the MGDG-AA preparation. The trend continues, and on Day 8, the peroxide value of the LCPUFA in the TG-AA oil is 280% of that in the MGDG-AA Preparation. See Table 9. This significant improvement of the oxidative stability of the LCPUFA in the MGDG-AA Preparation creates a novel product and method that can be used in nutritional product applications. The MGDG-AA Preparation presents the AA and other LCPUFA in a form that they are more stable from the degradation by oxidation at the elevated temperature and in an open air environment. The derivatization of LCPUFA triglyceride to mono and diglyceride forms provide a means to preserve the nutritional values and prolong the shelf-life of nutritional products containing these valuable fatty acids. By presenting the AA and other LCPUFA in the MGDG form, the potential detrimental harmful effects which are caused by the oxidation products of these valuable and yet labile fatty acids can be greatly reduced.

TABLE 9
Determination of Peroxide Value (meq/Kg) of TG-AA Oil and MGDG-
AA Preparation at 40° C. and Open Air

| | | MGDG-AA |
|---|---|---|
| Day | TG-AA Oil | (nonRBD) |
| 0 | 1.60 | 7.26 |
| 1 | 2.72 | 9.39 |
| 2 | 9.08 | 12.88 |
| 3 | 19.95 | 15.74 |
| 4 | 34.08 | 18.50 |
| 5 | 49.12 | 21.98 |
| 6 | 64.74 | 25.89 |
| 7 | 83.11 | 29.68 |
| 8 | 101.96 | 34.19 |

2.4) Improved Emulsification (Similar to Section 1.4)

Mono and diglycerides are amphiphilic lipids, having both hydrophilic and lipophilic properties which enable them to act as surface active agents and facilitate the interface of oil and water. Mono and diglyceride of soybean oil play important roles in stabilizing the matrices containing oil in water, such as those found in infant formula, nutritional supplements, and medical nutritional products. The MGDG-AA Preparation as shown in this invention containing approximately 84% of amphiphilic lipid molecules as shown in Table 8, such that they not only can provide an oxidative stable molecule to supply the LCPUFA in the nutritional products, but also can facilitate a more stable matrix for the finished products. The improvement of the emulsion enhances the physical stability, and gives rise to longer shelf-life of the products.

2.5) Improved Absorption (Similar to 1.5)

Approximately 90% of dietary fat is consumed as triglycerides. The triglycerides in the diet are first hydrolyzed by lingual and pancreatic lipases to fatty acid and mono and diacylglycerides before absorption. The pancreatic lipase is a 1,3-specific lipase, that is it cleaves fatty acyl groups from the positions 1 and 3 of triglycerides at rates many times greater than that for the 2-acyl groups. The majority of fatty acids esterified at the 2 position of the triglycerides are absorbed as 2-monoglycerides (2-MG). The presentation of LCPUFA as mono and diglyceride may reduces the needs of the hydrolysis action by the lipases. Especially for individuals that are deficient or compromised in the lipase activities, fatty acids fed in the mono and diglycerides forms can be absorbed easier and in a faster rate since no hydrolysis by the lipases is required.

The pancreatic lipase activity is generally not efficient for LCPUFA, such as AA. The MGDG-AA Preparation created from this invention is analyzed to determine the positional distribution of the AA on the glycerol backbone. The fatty acid profiles in the 1-MG and the 2-MG of the MGDG-AA Preparation are analyzed and the results are displayed in Table 10. The AA is found to be predominantly esterified in the 2-MG molecules. The AA represents 66.2% of the fatty acid in the 2-MG molecules, and only represents 23.6% of the fatty acid in the 1-MG molecules. Although the 1-MG is more abundant than the 2-MG in the preparation, as shown in Table 11, the overall percentage of total AA in the 2-MG is 26.17% whereas only 17.45% of total AA is in the 1-MG molecules. See Table 12. The AA in the 2-MG is 150% of that in the 1-MG. Based on the positional distribution of the AA in the MGDG-AA prepared according to this invention, the absorption of the AA present in this preparation can be superior, easier and at a faster rate, to those present in the triglyceride form. (See Section 1.5)

TABLE 10
Relative Fatty Acid Profile of 1-MG and 2-MG
of MGDG Preparation of High Arachidonic Acid (MGDG-AA)

| | AA 1-MG | AA 2-MG |
|---|---|---|
| C16:0 | 19.7 | 2.6 |
| C18:0 | 14.6 | 2.5 |
| C18:1n−9 | 10.5 | 3.4 |
| C18:2n−6 | 13.2 | 4.8 |
| C18:3n−6 | 2.9 | 1.8 |
| C20:0 | 1.0 | — |
| C20:2n−6 | 0.8 | 0.6 |
| C20:3n−6 | 3.0 | 4.2 |
| C20:4n−6 | 23.6 | 66.2 |
| C22:0 | 2.8 | 2.5 |
| C22:4n−6 | — | 1.1 |
| C24:0 | 5.2 | 10.3 |
| Others | 2.9 | — |
| Total | 100.0 | 100.0 |

TABLE 11
Positional Distribution of Mono- and Diglycerides Preparation of High
AA Oil

| | MGDG-AA |
|---|---|
| | % |
| Total MG | 37.89 |
| 1-MG (Rel to Total MG) | 66.44 |
| 2-MG (Rel to Total MG) | 35.56 |
| MG Ratio (1-MG/2-MG) | 1.87 |
| Total DG | 46.13 |
| 1,3-DG(Rel to Total DG) | 64.37 |
| 1,2-DG(Rel to Total DG) | 35.63 |
| DG Ratio (1,3-/1,2-) | 1.81 |
| DG/MG | 1.22 |

TABLE 12
Distribution of AA in Neutral Lipid Classes of MGDG-AA

| | % |
|---|---|
| 1-MG | 17.45 |
| 2-MG | 26.17 |
| FFA | 0.05 |
| 1,2-DG | 16.67 |
| 1,3-DG | 34.11 |
| TG | 5.55 |
| Total | 100.00 |

### Example 3

### Preparation of Mono and Diglycerides According to the Invention

3.1) Preparation of Mixture of High Arachidonic Acid Oil and Soy Oil (TG-AASOY)

High AA triglyceride oil, TG-AA, having the fatty acid profile as shown in Table 7, is obtains as in example 2.1. Soy Oil is obtained as commercial raw ingredient in the open market, for instance from EG Cargill, ADM, and the like. A mixture of 50:50 by weight of the TG-AA oil and the Soy Oil is prepared by weighing 50 g each of the two oils into a bottle and mixing thoroughly. The fatty acid profile of the resultant mixture is shown in Table 13.

TABLE 13
Fatty Acid Profile of AA/Soy 50/50 Mixture Triglyceride
Oil (TG-AASOY) Free Fatty Acid (FFA-AASOY) and
Mono- Diglycerides (MGDG-
AASOY) Preparations

| | TG-AASOY | FFA-AASOY | MGDG-AASOY |
|---|---|---|---|
| | Starting Oil | Preparation | Preparation |
| Fatty Acid | % | % | % |
| C16:0 | 11.02 | 11.14 | 11.48 |
| C18:0 | 5.90 | 5.98 | 6.90 |
| C18:1n−9 | 15.90 | 16.16 | 16.42 |
| C18:2n−6 | 31.34 | 31.63 | 31.06 |
| C18:3n−6 | 1.28 | 1.25 | 1.19 |
| C18:3n−3 | 3.75 | 3.68 | 3.43 |
| C20:0 | 0.62 | 0.64 | 0.67 |
| C20:3n−6 | 1.71 | 1.69 | 1.66 |
| C20:4n−6 | 19.92 | 18.98 | 18.07 |
| C22:0 | 1.83 | 1.90 | 1.92 |
| C24:0 | 4.47 | 4.49 | 4.58 |
| Others | 2.26 | 2.47 | 2.62 |
| Total | 100.00 | 100.00 | 100.00 |

3.2) Preparation of Free Fatty Acids of Mixture of High Arachidonic Acid Oil and Soy Oil (FFA-AASOY Preparation)

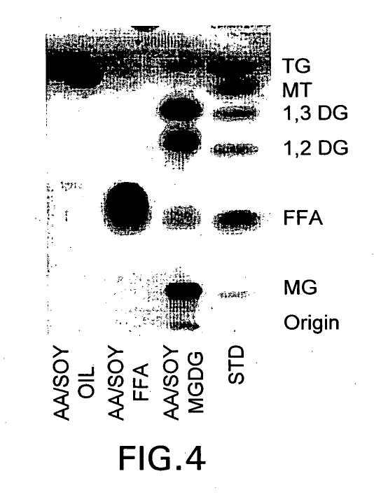

The FFA preparation of this oil mixture is prepared using the procedure similar to that described in Section 1.1) above. The total yield of the free fatty acid from 150 g of TG-AASOY triglyceride oil is 132 g, 93% of theoretical yield. The fatty acid profile of the FFA-AASoy Preparation is displayed in Table 13. The fatty acid profile of the FFA-AASOY Preparation is similar to that of the TG-AASOY starting material. The FFA-AASOY Preparation contains of exclusively FFA, and no trace of triglyceride from the starting oil is found in the extract. See FIG. 4 and Table 14.

TABLE 14
Neutral Lipid Classes Composition of AA/Soy Mixture,
Triglyceride Oil (TG-AASOY), Free Fatty Acid
(FFA-AASOY) and Mono- Diglyceride (MGDG-
AASOY) Preparations

| | TG-AASOY | FFA-AASOY | MGDG-AASOY |
|---|---|---|---|
| | Starting Oil | Preparation | Preparation |
| | % | % | % |
| Triglyceride | 100 | ND | 4.43 |
| Diglyceride | ND | ND | 48.33 |
| Monoglyceride | ND | ND | 38.69 |
| Free Fatty | ND | 100 | 8.55 |
| Acid | | | |

3.3) Preparation of AA/Soy Mono and Diglycerides

The FFA preparation of the AA Soy mixture (FFA-AASOY), 132 g, is used to prepare the mono and diglycerides using the procedure described in Section 1.2) above. Mixed tocopherols, 0.66 g, or 0.5% of the free fatty acid by weight, is added into the FFA-AASOY preparation. Distilled monoglyceride, 1.32 g, or 1% of the FFA by weight, is added prior to the esterification. Catalyst, p-toluene sulfonic acid, 20-30 mg is added into the individual tubes. A total of 142 g of MGDG-AASOY preparation are obtained from this process. The yield is calculated to be 96% of theoretical. The fatty acid profile of the MGDG-AASOY preparation is similar to that of the FFA preparation (FFA-AASOY) and the TG-AASOY starting oil. See Table 13. The preparation contains 38.69% of monoglyceride, 48.33% of diglyceride, 4.43% of triglyceride, and 8.55% of FFA. See FIG. 4 and Table 14.

### Example 4

### Preparation of Mono and Diglycerides According to the Invention

4.1) Preparation of Freeze Dried Egg Yolk Powder (EYP)

Chicken eggs enriched with AA are purchased from EggsAActly Eggs (3145 Stoney Bridge Lane, Columbus, Ohio 43221-4913). The eggs are hard boiled and peeled. Egg yolk is isolated from the egg white and freeze dried using a commercial free drier (Hull Freeze Drier, Model 72F100, or equivalent). The egg yolk powder is then stored in freezer until used.

4.2) Preparation of Free Fatty Acids of Egg Yolk Powder (FFA-EYP Preparation)

No extraction of total lipid from the egg yolk powder (EYP) is required. The fatty acids in the EYP are primarily present in triglyceride (about 80%) and phospholipids (about 20%) forms. The fatty acid profile of the egg yolk powder is shown in Table 15. The FFAs are prepared from the freeze dried egg yolk powder as follows. Approximately 1.2 g of the egg yolk powder is placed in a 50 ml screw cap tube. Ethanolic potassium hydroxide, 0.5N, 15 ml is added into the egg yolk powder. The sample is heated in a boiling water bath for 1 hour. After heating, the sample is cooled to room temperature and 20 ml of lab water is added and mixed. The sample is transferred into a separatory funnel. Approximately 1.5 ml of concentrated hydrochloric acid is added and mixed. The pH of the content is tested using a pH paper. The pH is adjusted to 2-3 by adding concentrated hydrochloric acid drop wise. The mixture is extracted twice with 20 ml of hexane. The hexane extracts are combined and wash thrice with 20 ml of saturated sodium chloride solution. The pH of the last water wash is tested and found to be neutral, i.e. pH 7. The hexane extract is dried over anhydrous sodium sulfate for >60 minutes. The hexane extract is decanted to a preweighed 50 ml screw cap tube. The sample is evaporated to dryness under nitrogen. The final sample is dissolved in 40 ml of methylene chloride. Ten ml of this solution is retained for fatty acid analysis. To 30 ml of this solution, 4.5 mg of mixed tocopherols is added into the solution. The FFA prepared by this procedure is analyzed using thin layer chromatography, and the result indicated that only free fatty acid is found in the preparation, and no trace of phospholipid or triglyceride is present in this preparation. The fatty acid profile of the FFA-EYP Preparation is similar to that of the total egg yolk powder. See Table 15.

TABLE 15
Fatty Acid Profile of Enriched AA Egg Yolk Powder
(EYP) Free Fatty Acid of EYP (FFA-EYP) and
Mono- Diglycerides (MGDG-EYP) Preparations

| | EYP | FFA-EYP | MGDG-EYP |
|---|---|---|---|
| | Powder | Preparation | Preparation |
| Fatty Acid | % | % | % |
| C 16:0 | 22.30 | 21.91 | 22.32 |
| C 16:1 | 1.51 | 1.45 | 1.30 |
| C 18:0 | 10.76 | 10.45 | 15.40 |
| C 18:1 | 30.04 | 29.59 | 28.52 |
| C 18:2n6 | 25.15 | 24.52 | 22.38 |
| C 18:3n6 | 1.47 | 1.51 | 1.25 |
| C 18:3n3 | 0.48 | 0.52 | 0.38 |
| C 20:1n9 | 0.84 | 0.85 | 0.82 |
| C 20:3n−6 | 0.84 | 0.81 | 0.83 |
| C 20:4n−6 | 3.38 | 3.12 | 3.35 |
| C 22:6n−3 | 1.02 | 0.83 | 0.70 |
| Others | 2.21 | 0.00 | 0.00 |
| Total | 100.00 | 100.00 | 100.00 |

4.3) Preparation of Mono and Diglycerides of Fatty Acid from Egg Yolk Powder (MGDG-EYP Preparation)

Ten ml of the methylene chloride solution of the free fatty acid preparation obtained is pipetted into a 50 ml screw cap tube. Distilled monoglyceride, 9 mg, is added into the tube. The sample is then evaporated to dryness under nitrogen. Glycerol (1,2,3-propanetriol, Sigma Aldrich Chemical Co.) 14 ml is added into the tube. Catalyst, p-toluene sulfonic acid 20 mg and a small stir bar are added into the tube and the tube is capped tight. The sample is heated at 120° C. for 8 hours with constant stirring during the heating. At the end of the heating, it is cooled to room temperature and 30 ml of water is added and mixed. The sample is transferred to a separatory funnel and extracted twice with 20 ml of ethyl ether. The ether extracts are combined, washed once with 40 ml of lab water, and dried over anhydrous sodium sulfate. The monoglyceride (MG), diglyceride (DG), triglyceride (TG), and FFA in this sample are quantitated and the results are displayed in Table 16. The MG, DG, TG and FFA in this sample are found to be 42.27%, 52.44%, 4.36%, and 0.93%, respectively. The total mono and diglycerides concentration in this preparation is over 94%. The results demonstrate that the procedure is applicable in preparing MGDG from the egg yolk powder without extraction of lipid from the powder. The procedure converts the fatty acids from the phospholipids and triglycerides into free unesterified fatty acids. The fatty acid profile of the MGDG-EYP is similar to those in the FFA-EYP and the original EYP starting material. See Table 15.

TABLE 16
Neutral Lipid Classes Composition of Egg Yolk Powder
(EYP) Free Fatty Acid of EYP (FFA-EYP) and
Mono- Diglycerides (MGDG-EYP)
Preparations

| | EYP | FFA-EYP | MGDG-EYP |
|---|---|---|---|
| | Powder | Preparation | Preparation |
| | % | % | % |
| Triglyceride | NA | ND | 4.36 |
| Diglyceride | NA | ND | 52.44 |
| Monoglyceride | NA | ND | 42.27 |
| Free Fatty | NA | 100 | 0.93 |
| Acid | | | |

NA = Not Analyzed
ND = Not Detected

### Example 5

### Preparation of Mono and Diglyceride According to the Invention

5.1) Preparation of Free Fatty Acids of Fungal and Algal Oils with AA and DHA (FFA-ARASCO and FFA-DHASCO Preparations)

Fungal and Algal Oils with AA and DHA, trade names, ARASCO and DHASCO, are purchased from Martek Bioscience Corporation (555 Rolling Hills Lane, Winchester, Ky., 40391). The fatty acid profiles of these two triglyceride oils are displayed in Table 17. Approximately 1.2 g each of TG-ARASCO and TG-DHASCO are weighed into two separate 50 ml screw cap tubes. The samples are treated similar to the procedure described in Example 4 above. The final FFAs liberated are dissolved in 40 ml of methylene chloride. Ten ml of each of the preparation is retained for thin layer chromatography analysis. To thirty ml of each of the preparation, 4.5 mg of mixed tocopherols are added and used for the preparation of mono- and diglycerides. The fatty acid profile of the FFA-ARASCO and FFA-DHASCO Preparations are summarized in Tables 17 and 18. The fatty acid profiles of the FFA-ARASCO and FFA-DHASCO Preparations are similar to those of the starting triglyceride oils, TG-ARASCO and TG-DHASCO. No trace of triglyceride is detected in these free fatty acid preparations, FFA-ARASCO and FFA-DHASCO. See Tables 19 and 20.

TABLE 17
Fatty Acid Profile of ARASCO Tryglyceride Oil (TG-ARASCO),
Free Fatty Acid (FFA-ARASCO) and Mono- Diglycerides
(MGDG-ARASCO)

| | TG-ARASCO | FFA-ARASCO | MGDG-ARASCO |
|---|---|---|---|
| | Starting Oil | Preparation | Preparation |
| Fatty Acid | % | % | % |
| C 14:0 | 1.45 | 1.46 | 1.44 |
| C 16:0 | 13.89 | 13.97 | 14.67 |
| C 18:0 | 11.18 | 11.25 | 14.41 |
| C 18:1n9 | 12.56 | 12.65 | 12.74 |
| C 18:2n6 | 7.29 | 7.35 | 7.07 |
| C 18:3n6 | 3.32 | 3.26 | 3.01 |
| C 20:0 | 0.91 | 0.95 | 1.04 |
| C 20:1n9 | 0.37 | 0.38 | 0.39 |
| C 20:3n−6 | 3.47 | 3.41 | 3.36 |
| C 20:4n−6 | 39.92 | 37.32 | 36.01 |
| C 20:5n−3 | 0.11 | 0.17 | 0.11 |
| C 22:0 | 1.63 | 1.80 | 1.76 |
| C 24:0 | 1.75 | 1.74 | 1.72 |
| C 24:1n−9 | 0.14 | 0.14 | 0.14 |
| Others | 2.01 | 4.15 | 2.13 |
| Totals | 100.00 | 100.00 | 100.00 |

TABLE 18L
Fatty Acid Profile of DHASCO Triglyceride Oil (TG-DHASCO),
Free Fatty Acid (FFA-DHASCO) and Mono- and Diglycerides
(MGDG-DHASCO)

| | TG-DHASCO | FFA-DHASCO | MGDG-DHASCO |
|---|---|---|---|
| | Starting Oil | Preparation | Preparation |
| Fatty Acid | % | % | % |
| C 12:0 | 1.91 | 2.08 | 1.52 |
| C 14:0 | 13.74 | 14.94 | 14.19 |
| C 16:0 | 16.84 | 18.33 | 18.72 |
| C 16:1 | 1.19 | 1.29 | 1.23 |
| C 18:0 | 0.90 | 0.99 | 4.07 |
| C 18:1n9 | 22.16 | 24.11 | 23.93 |
| C 18:2n6 | 0.64 | 0.70 | 0.70 |
| C 22:6n−3 | 41.16 | 36.18 | 34.59 |
| Others | 1.46 | 1.39 | 1.05 |
| Totals | 100.00 | 100.00 | 100.00 |

TABLE 19
Neutral Lipid Classes Composition of ARASCO Triglyceride
Oil (TG-ARASCO), Free Fatty Acid (FFA-ARASCO) and
Mono- Diglycerides (MGDG-ARASCO)

| | TG-ARASCO | FFA-ARASCO | MGDG-ARASCO |
|---|---|---|---|
| | Starting Oil | Preparation | Preparation |
| | % | % | % |
| Triglyceride | 100 | ND | 4.45 |
| Diglyceride | ND | ND | 47.82 |
| Monoglyceride | ND | ND | 46.26 |
| Free Fatty Acid | ND | 100 | 1.47 |

ND = Not Detected

TABLE 20
Neutral Lipid Classes Composition of DHASCO Triglyceride
Oil (TG-DHASCO), Free Fatty Acid (FFA-DHASCO)
and Mono- and Diglycerides
(MGDG-DHASCO)

| | TG-DHASCO | FFA-DHASCO | MGDG-DHASCO |
|---|---|---|---|
| | Starting Oil | Preparation | Preparation |
| | % | % | % |
| Triglyceride | 100 | ND | 5.23 |
| Diglyceride | ND | ND | 49.00 |
| Monoglyceride | ND | ND | 44.57 |
| Free Fatty Acid | ND | 100 | 1.20 |

ND = Not Detected

5.2) Preparation of Mono and Diglycerides of Fatty Acids of Fungal and Algal Oils with AA and DHA (MGDG-ARASCO and MGDG-DHASCO Preparations)

Ten ml of the preparations FFA-ARASCO and FFA-DHASCO containing 0.5% of mixed tocopherols are pipetted into two separate 50 ml screw cap tubes. The amount of FFA in each tube is approximately 300 mg. Distilled monoglyceride of hardened soy oil, 9 mg, is added and the samples to facilitate the mixing of glycerol and the FFA. The samples are evaporated to dryness under nitrogen. The samples are treated in the same manner as described in Section 4.3) described above.

The fatty acid profile of the MGDG-ARASCO and MGDG-DHASCO Preparations are similar to those in the FFA-ARASCO and FFA-DHASCO Preparations. See Tables 17 and 18. The neutral lipid classes, i.e., MG, DG, TG, and FFA in these samples are quantitated and the results are displayed in Tables 19 and 20. The MG, DG, TG and FFA in MGDG-ARASCO samples are 46.26%, 47.82%, 4.45%, and 1.47%, respectively. The MG, DG, TG and FFA in MGDG-DHASCO sample are 44.57%, 49.00%, 5.23%, and 1.20%, respectively. The mono and diglycerides concentrations for both preparations are 93-94%. The results demonstrate that the procedure described in this invention is applicable in preparing MGDG using TG-ARASCO and TG-DHASCO as starting materials.

### Example 6

### Preparation of Mono and Diglycerides According to the Invention

6.1) Preparation of Free Fatty Acids of Salmon, Cod Liver, and Borage Oils (FFA-Salmon, FFA-CLO, and FFA-Borage Preparations)

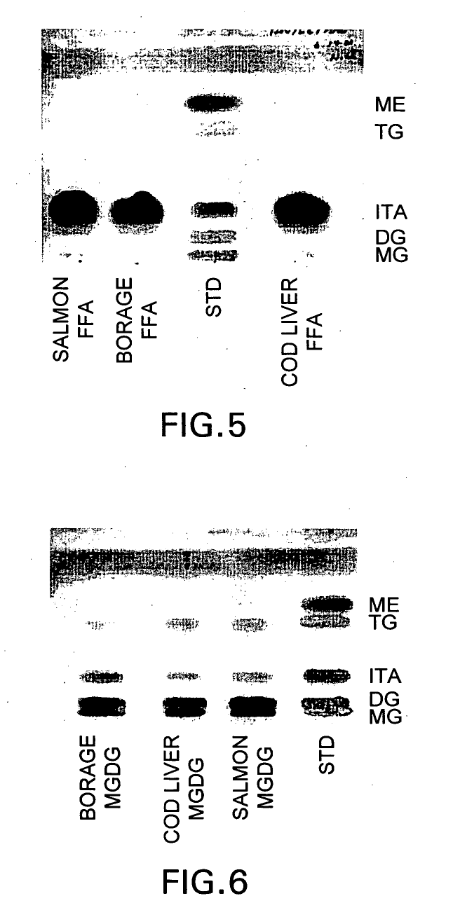

The Salmon and Cod Liver oils are obtained from Maritex (M.P. Bruuns Gade 27, DK-8000, Aarhus, Netherland) and the Borage Oil is a product of PGE Canada (118 Veterinary Road, Suite 5000, Saskatoon, SK, S7N 2R4, Canada). The fatty acid profiles of these three triglyceride oils, TG-Salmon, TG-CLO and TG-Borage, are shown in Tables 21, 23 and 25. Approximately 2.5 g of each of the oils, TG-Salmon, TG-CLO and TG-Borage, are pipetted into 9 screw cap tubes. Mixed tocopherols, 10 mg, is added into each tube. Thirty ml of 0.5N KOH in ethanol is added to each tube and tightly capped. The tubes are heated in a boiling water bath for one hour. The tubes are then cooled to room temperature. Samples from the 9 tubes for each oil are combined in a 1-liter separatory funnel, and 270 ml of laboratory water is added and mixed well. Approximately 14 ml of concentrated hydrochloric acid is added to each sample, mixed, and the pH is checked using pH paper. The pH of each sample is 2-3. Each sample is extracted twice with 270 ml of EE. The ethyl ether extracts of each sample is combined and washed thrice with equal volume of laboratory water. The FFA preparations from each sample are weighed after the EE solvent are evaporated. The total FFA-Salmon, FFA-CLO, and FFA-Borage Preparations obtained are 20.5 g, 19.8 g, and 19.2 g, respectively. The FFA preparations are analyzed using thin layer chromatography, and the results clearly indicated that no triglycerides from the starting material are found in the FFA preparations. See FIG. 5 and Tables 22, 24, and 26. The fatty acid profiles of the FFA preparations of these three oils are also analyzed and summarized in Tables 21, 23 and 25. The results show that the fatty acid profiles of the FFA preparations of the Salmon, Cod-Liver, and Borage oils are practically identical to those of the starting triglyceride oils.

TABLE 21
Fatty Acid Profile of Salmon Oil (TG-Salmon) Free Fatty Acid (FFA-
Salmon), and Mono-Diglycerides (MGDG-Salmon) Preparations

| | TG-Salmon | FFA-Salmon | MGDG-Salmon |
|---|---|---|---|
| | Starting Oil | Preparation | Preparation |
| Fatty Acid | % | % | % |
| C14:0 | 4.95 | 5.00 | 4.96 |
| C16:0 | 11.90 | 12.13 | 12.62 |
| C16:1n-7 | 5.94 | 6.03 | 5.66 |
| C18:0 | 2.30 | 2.36 | 4.82 |
| C18:1n-9 | 18.74 | 19.22 | 19.13 |
| C18:1n-7 | 2.99 | 3.09 | 3.19 |
| C18:2n-6 | 6.92 | 7.10 | 6.74 |
| C18:3n-3 | 1.89 | 1.90 | 1.68 |
| C18:4n-3 | 2.34 | 2.28 | 1.87 |
| C20:1n-9 | 8.76 | 9.06 | 9.24 |
| C20:2n-6 | 0.55 | 0.57 | 0.57 |
| C20:4n-3 | 1.55 | 1.51 | 1.39 |
| C20:5n-3 | 6.62 | 6.22 | 5.47 |
| C22:1n-9 | 8.69 | 8.92 | 9.12 |
| C22:5n-3 | 2.88 | 2.72 | 2.60 |
| C22:6n-3 | 8.82 | 7.84 | 7.26 |
| C24:1n-9 | 0.73 | 0.75 | 0.77 |
| Others | 3.43 | 3.28 | 2.91 |
| Total | 100.00 | 100.00 | 100.00 |

TABLE 22
Neutral Lipid Classes Composition of Salmon (TG-Salmon)
Free Fatty Acid (FFA-Salmon), and Mono-Diglycerides
(MGDG-Salmon) Preparations

| | TG-Salmon | FFA-Salmon | MGDG-Salmon |
|---|---|---|---|
| | Starting Oil | Preparation | Preparation |
| | % | % | % |
| Triglyceride | 100 | ND | 6.41 |
| Diglyceride | ND | ND | 48.27 |
| Monoglyceride | ND | ND | 39.65 |
| Free Fatty Acid | ND | 100 | 6.73 |

ND: Not Detected

TABLE 23
Fatty Acid Profile of Cod Liver Oil (TG-CLO) Free Fatty Acid (FFA-
CLO), and Mono-Diglycerides (MGDG-CLO) Preparations

| | TG-CLO | FFA-CLO | MGDG-CLO |
|---|---|---|---|
| | Starting Oil | Preparation | Preparation |
| Fatty Acid | % | % | % |
| C14:0 | 4.22 | 4.35 | 4.25 |
| C16:0 | 10.71 | 11.07 | 11.48 |
| C16:1n-7 | 6.44 | 6.63 | 6.14 |
| C18:0 | 2.17 | 2.24 | 4.72 |
| C18:1n-9 | 18.03 | 18.63 | 18.46 |
| C18:1n-7 | 3.90 | 4.05 | 4.12 |
| C18:2n-6 | 1.98 | 2.07 | 2.04 |
| C18:3n-3 | 1.28 | 1.32 | 1.10 |
| C18:4n-3 | 3.00 | 2.94 | 2.40 |
| C20:1n-9 | 13.32 | 13.74 | 13.89 |
| C20:4n-3 | 0.80 | 0.76 | 0.78 |
| C20:5n-3 | 8.87 | 8.26 | 7.32 |
| C22:1n-9 | 8.11 | 8.36 | 8.39 |
| C22:5n-3 | 1.08 | 1.04 | 1.00 |
| C22:6n-3 | 12.18 | 10.70 | 10.08 |
| Others | 3.91 | 3.84 | 3.83 |
| Total | 100.00 | 100.00 | 100.00 |

TABLE 24
Neutral Lipid Classes Composition of Cod Liver (TG-CLO) Free Fatty
LAcid (FFA-CLO), and Mono-Diglycerides (MGDG-DLO) Preparations

| | TG-CLO | FFA-CLO | MGDG-CLO |
|---|---|---|---|
| | Starting Oil | Preparation | Preparation |
| | % | % | % |
| Triglyceride | 100 | ND | 6.61 |
| Diglyceride | ND | ND | 49.26 |
| Monoglyceride | ND | ND | 38.75 |
| Free Fatty Acid | ND | 100 | 5.45 |

TABLE 25
Fatty Acid Profile of Borage Oil (TG-Borage) Free Fatty Acid
(FFA-Borage), and Mono- Diglycerides
(MGDG-Borage) Preparations

| | TG-Borage | FFA-Borage | MGDG-Borge |
|---|---|---|---|
| | Starting Oil | Preparation | Preparation |
| Fatty Acid | % | % | % |
| C16:0 | 10.58 | 10.70 | 11.05 |
| C18:0 | 4.21 | 4.32 | 6.59 |
| C18:1n-9 | 17.35 | 17.55 | 17.50 |
| C18:1n-7 | 0.63 | 0.66 | 0.76 |
| C18:2n-6 | 36.06 | 35.90 | 34.40 |
| C18:3n-6 | 20.66 | 19.87 | 18.50 |
| C20:1n-9 | 4.06 | 4.18 | 4.31 |
| C22:1n-9 | 2.68 | 2.74 | 2.76 |
| C24:1n-9 | 1.71 | 1.75 | 1.76 |
| Others | 2.17 | 2.33 | 2.37 |
| Total | 100.10 | 100.00 | 100.00 |

TABLE 26
Neutral Lipid Classes Composition of Borage Oil (TG-Borage)
Free Fatty Acid (FFA-Borage), and Mono- Diglycerides
(MGDG-Borage) Preparations

| | TG-Borage | FFA-Borage | MGDG-Borage |
|---|---|---|---|
| | Starting Oil | Preparation | Preparation |
| | % | % | % |
| Triglyceride | 100 | ND | 5.00 |
| Diglyceride | ND | ND | 44.99 |
| Monoglyceride | ND | ND | 38.80 |
| Free Fatty Acid | ND | 100 | 11.26 |

6.2) Preparation of Mono and Diglycerides of Fatty Acids of Salmon, Cod Liver and Borage Oils (MGDG-Salmon, MGDG-CLO, and MGDG-Borage Preparations)

The FFA-Salmon, FFA-CLO, and FFA-Borage Preparations are used to prepare MGDG-Salmon, MGDG-CLO and MGDG-Borage using the procedure similar to that described in Section 1.2) above. To each of the FFA-preparations, 0.1 g of mixed tocopherols and 0.6 g of commercial distilled monoglyceride from hardened soy oil is added to the FFA preparations. The final concentrations of the mixed tocopherols and distilled monoglyceride are 0.5% and 3% w/w of the FFA, respectively. A total of approximately 7.2 g of each of the FFA preparation are used to prepare the MGDG preparations as follows. Approximately 0.3 g of the FFA containing tocopherols and distilled monoglyceride mixture is pipetted into each of the 24 screw cap tubes. The solvent is evaporated under nitrogen. Catalyst, p-toluene sulfonic acid, 20-30 mg is added. Approximately 14 ml of the glycerin and a small stir bar are added to each tube. The tubes are flushed with nitrogen and capped tight. They are heated at 120° C. for 5-6 hours. The preparations are tested for the formation of mono- and diglycerides, and the concomitant disappearance of FFA. See FIG. 6. The resulting MGDG-Preparations of the three oils are extracted, washed, dried, and weighed similar to that described in Section 1.2). The weights of the MGDG-Salmon, MGDG-CLO, and MGDG-Borage preparations are 7.52 g, 6.83 g, and 5.92 g, respectively. The neutral lipid classes of the MGDG-Salmon, MGDG-CLO, and MGDG-Borage preparations are analyzed and the results are displayed in Tables 22, 24 and 26. The MG, DG, TG, and FFA of MGDG-Salmon are 39.65%, 48.27%, 6.41% and 6.73%, respectively. See Table 22. The MG, DG, TG, and FFA of MGDG-CLO are 38.76%, 49.26%, 5.45%, and 6.61%, respectively. See Table 24. The MG, DG, TG, and FFA of MGDG-Borage are 38.80%, 44.99%, 5.00%, and 11.26%, respectively. See Table 26. The mono- and diglycerides in these preparations ranged from 83-88%. The fatty acid composition of the MGDG preparations of these three oils are summarized in Tables 21, 23 and 25. The fatty acid profile of the MGDG preparations are similar to those of the FFA-preparations and the TG-oils of the starting materials.

While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. A glyceride composition, comprising:
from about 10 to about 90% by weight of monoglycerides; and
from about 10 to about 90% by weight of diglycerides, wherein at least about 20% by weight of the combined monoglycerides and diglycerides comprise monoglycerides and diglycerides of long chain polyunsaturated fatty acids.

2. The glyceride composition of claim 1, wherein at least about 20% by weight of the monoglycerides and diglycerides comprise monoglycerides and diglycerides of polyunsaturated fatty acids having at least about 20 carbon atoms and at least about 3 carbon-carbon double bonds.

3. The glyceride composition of claim 1, wherein at least about 25% by weight of the monoglycerides and diglycerides comprise monoglycerides and diglycerides of long chain polyunsaturated fatty acids.

4. The glyceride composition of claim 1, wherein the monoglycerides and diglycerides comprise at least about 20% by weight of monoglycerides and diglycerides of one or more of DHA, AA, LA, EPA, ALA, and GLA.

5. The glyceride composition of claim 1, wherein a ratio of the monoglycerides to the diglycerides is from about 1:3 to about 10:1.

6. The glyceride composition of claim 1, wherein a ratio of the monoglycerides and diglycerides to triglycerides is from about 3:1 to about 100:1.

7. The glyceride composition of claim 1, further comprising from about 0.1 to about 10% by weight of at least one free fatty acid and wherein the glyceride composition comprises about 20% by weight or less of triglycerides.

8. A nutritional formulation comprising the glyceride composition of claim 1.

9. A method of making a glyceride composition, comprising:
hydrolyzing a mixture comprising triglycerides to provide free fatty acids, the free fatty acids comprising free long chain polyunsaturated fatty acids;
combining the free fatty acids with glycerol and heating at a temperature sufficient to esterify the free fatty acids, wherein a weight ratio of free fatty acids to glycerol is from about 1:5 to about 1:100; and
recovering the glyceride composition, the glyceride composition comprising from about 10 to about 90% by weight of monoglycerides, from about 10 to about 90% by weight of diglycerides, wherein at least about 20% by weight of the monoglycerides and diglycerides comprise monoglycerides and diglycerides of long chain polyunsaturated fatty acids.

10. The method of claim 9, wherein the weight ratio of free fatty acids to glycerol is from about 1:30 to about 1:80.

11. The method of claim 9, wherein the glyceride composition comprises about 10% by weight or less of triglycerides.

12. The method of claim 9, wherein the mixture further comprises phospholipids comprising phospholipids of long chain polyunsaturated fatty acids, and the mixture is hydrolyzed with a hydroxide compound.

13. The method of claim 9, wherein at least one of an antioxidant and an emulsifier are further combined with the free fatty acids and glycerol, and the free fatty acids are esterified under a temperature from about 70° C. to about 150° C.

14. The method of claim 9, wherein the free fatty acids are esterified using a catalyst.

15. The method of claim 9, further comprising at least one of deodorizing, refining, and bleaching the glyceride composition.

16. A nutritional formulation, comprising:
proteins; carbohydrates; and fats
wherein said fats include a glyceride composition comprising from about 10 to about 90% by weight of monoglycerides, from about 10 to about 90% by weight of diglycerides, wherein at least about 20% by weight of the monoglycerides and diglycerides comprise monoglycerides and diglycerides of long chain polyunsaturated fatty acids.

17. The nutritional formulation of claim 16, wherein at least about 25% by weight of the monoglycerides and diglycerides in the glyceride composition comprise monoglycerides and diglycerides of long chain polyunsaturated fatty acids.

18. The nutritional formulation of claim 16, wherein the fats further comprise from about 1 to about 20% by weight of triglycerides.

19. The nutritional formulation of claim 16, wherein the fats comprise from about 2 to about 50% by weight of said glyceride composition.

20. The nutritional formulation of claim 16, wherein the fats comprise from about 5 to about 90% by weight of said glyceride composition.

21. A method of delivering long chain polyunsaturated fatty acids to a human, comprising:
administering to the human an effective amount of a glyceride composition comprising from about 10 to about 90% by weight of monoglycerides, from about 10 to about 90% by weight of diglycerides, wherein at least about 20% by weight of the monoglycerides and diglycerides comprise monoglycerides and diglycerides of long chain polyunsaturated fatty acids.

22. The method of claim 21, wherein the human is an infant.

23. The method of claim 21, wherein the glyceride composition is a component in a nutritional formulation.

24. A glyceride composition, comprising:
from about 20 to about 80% by weight of monoglycerides; and
from about 20 to about 80% by weight of diglycerides, wherein at least about 25% by weight of the monoglycerides and diglycerides comprise monoglycerides and diglycerides of long chain polyunsaturated fatty acids selected from the group consisting of one or more of DHA, AA, LA, EPA, ALA, and GLA.

25. The glyceride composition of claim 24, wherein at least about 20% by weight of the monoglycerides and diglycerides comprise monoglycerides and diglycerides of polyunsaturated fatty acids having at least about 20 carbon atoms and at least about 3 carbon-carbon double bonds.

26. The glyceride composition of claim 24, wherein at least about 30% by weight of the monoglycerides and diglycerides comprise monoglycerides and diglycerides of long chain polyunsaturated fatty acids.

27. The glyceride composition of claim 24 comprising from about 30 to about 70% by weight of monoglycerides and from about 30 to about 70% by weight of diglycerides, wherein the monoglycerides and diglycerides comprise at least about 30% by weight of at least one selected from the group consisting of monoglycerides and diglycerides of one or more of DHA, AA, LA, ALA, EPA, and GLA.
